(19) United States
(12) Patent Application Publication
(10) Pub. No.: US 2020/0087630 A1
(43) Pub. Date: Mar. 19, 2020

(54) INFLUENZA VIRUS AND TYPE 1 DIABETES

(71) Applicants: Ospedale San Raffaele S.r.l., Milano (IT); Istituto Zooprofilattico Sperimentale delle Venezie, Legnaro (Padova) (IT)

(72) Inventors: Lorenzo Piemonti, Carate Brianza (Monza Brianza) (IT); Ilaria Capua, Noventa Padovana (Padova) (IT)

(73) Assignees: Ospedale San Raffaele S.r.l., Milano (IT); Istituto Zooprofilattico Sperimentale delle Venezie, Legnaro (Padova) (IT)

(21) Appl. No.: 16/505,774

(22) Filed: Jul. 25, 2019

Related U.S. Application Data
(63) Continuation of application No. 15/862,773, filed on Jan. 5, 2018, now abandoned.
Application No. 15/862,773 is a continuation of application No. 14/435,016, filed on Apr. 10, 2015, now abandoned.

(30) Foreign Application Priority Data
Oct. 10, 2012 (GB) 1218195.4

(51) Int. Cl. C12N 7/00; A61K 45/06; A61K 31/351; A61K 31/215; A61K 39/12; C07K 14/005; A61K 39/145

(52) U.S. Cl. CPC C12N 7/00; A61K 45/06; A61K 31/351; A61K 31/215; C12N 2760/16122; C07K 14/005; A61K 39/145; C12N 2760/16134; C12N 2760/16133; A61K 39/12

(57) ABSTRACT

Type 1 diabetes mellitus is characterized by loss of pancreatic insulin-producing beta cells, resulting in insulin deficiency. The usual cause of this beta cell loss is autoimmune destruction. The inventors provide the first evidence of a causal link between influenza virus infection and the development of type 1 diabetes and/or pancreatitis. This causal link between infection and type 1 diabetes and/or pancreatitis provides various therapeutic, prophylactic and diagnostic opportunities.

### TECHNICAL FIELD

The present invention relates to the involvement of viruses in type 1 diabetes, and it is an object of the invention to provide further and improved materials and methods that can be used in the diagnosis, prevention, treatment and prognosis of type 1 diabetes in patient(s), particularly for children.

### BACKGROUND ART

Type 1 diabetes mellitus (previously known as IDDM) is characterized by loss of pancreatic insulin-producing beta cells, resulting in insulin deficiency. The usual cause of this beta cell loss is autoimmune destruction.

It has been proposed that the autoimmune destruction may be linked to a viral infection. For a virus to act as a trigger for autoimmune beta cell destruction, various mechanisms have been proposed. For instance, cytolytic infection of beta cells could occur, leading to their destruction and/or to the release of normally-sequestered antigens, which might then trigger pathogenic autoreactive T-cell responses. Alternatively, epitopes displayed by the virus may elicit auto-reactive antibodies and/or T cells, thereby providing the basis of autoimmunity.

The rapid worldwide increase in the incidence of Type 1 diabetes suggests a major role for environmental factors in its aetiology. According to cross-sectional and prospective studies on Type 1 diabetes patients and/or prediabetic individuals, virus infections may be one of these.

Various viruses have been linked to type 1 diabetes [1]. For instance, reference 2 noted in 2001 that 13 different viruses had been reported to be associated with its development in humans and in various animal models, including mumps virus, rubella virus, cytomegalovirus and coxsackie B virus.

### DISCLOSURE OF THE INVENTION

The inventors have for the first time identified a causal link between influenza A virus infection and type 1 diabetes. The inventors have also identified a causal link between influenza A virus infection and pancreatitis. Based on these causal links, the inventors conclude that in at least some cases, onset of Type 1 diabetes and/or pancreatitis is due to prior infection with influenza A virus, e.g., as a child.

Non-systemic influenza A viruses are the most common cause of influenza A infection in mammals and birds. Non-systemic influenza viruses are not usually found in internal organs.

Although previous studies have reported correlations between certain influenza A virus (IAVs) infections and pancreatic damage in mammals [3], none has established whether there exists a causal relationship [3,4]. Indeed, reference 5 inoculated mammals with influenza A virus and identified no influenza A virus antigen in the pancreas, and so the current opinion is that it is unlikely that influenza A virus infection is a direct cause of pancreatic damage.

Non-systemic influenza A viruses are able to replicate only in the presence of trypsin or trypsin-like enzymes, and so their replication is believed to be restricted to the respiratory and enteric tract. Indeed, none of the prior art has actually demonstrated that IA V are even able to grow in pancreatic cells, and no data are available on direct consequences of IAV replication in the pancreas. The inventors have demonstrated that surprisingly, non-systemic avian influenza A viruses cause severe pancreatitis resulting in a dismetabolic condition comparable with diabetes as it occurs in birds. The inventors have also found that human influenza A viruses are able to grow in human pancreatic primary cells and cell lines, showing a causal link between influenza A virus infection and type 1 diabetes and/or pancreatitis.

The identification of a direct causal link between influenza A virus infection and type 1 diabetes provides various opportunities for prevention, treatment, diagnosis and prognosis of type 1 diabetes. Similarly, the identification of a direct causal link between influenza A virus infection and pancreatitis provides various opportunities for prevention, treatment, diagnosis and prognosis of pancreatitis. At the time of administration of composition(s) of the invention, the patient is preferably a child. Administration of composition(s) of the invention to a patient (e.g., a child) thus helps prevent development of type 1 diabetes and/or pancreatitis later in the patient's life, e.g., as an adult. Similarly, diagnostic methods of the invention are performed on samples obtained from a patient (e.g., a child) to determine, e.g., whether the patient has a predisposition for developing type 1 diabetes and/or pancreatitis later in life, e.g., as an adult. The invention therefore provides an immunogenic composition comprising an influenza A virus immunogen for use in preventing or treating type 1 diabetes and/or pancreatitis in a patient, preferably a child. The invention also provides a composition comprising an antiviral compound effective against an influenza A virus for use in preventing or treating type 1 diabetes and/or pancreatitis in a patient, preferably a child. The invention also provides an immunogenic composition comprising an influenza A virus immunogen and an antiviral compound for use in preventing or treating type 1 diabetes and/or pancreatitis in a patient, preferably a child. In some embodiments, the composition further comprises an immunomodulatory compound effective to inhibit natural killer cell activity. In some embodiments the composition further comprises a pharmaceutically acceptable carrier.

In some embodiments, the composition is a vaccine composition, optionally further comprising an adjuvant, preferably an oil-in-water emulsion. In some embodiments, the composition is for use as a pharmaceutical.

The invention also provides a method for preventing or treating type 1 diabetes and/or pancreatitis in a patient, comprising a step of administering to the patient a composition of the invention.

In some embodiments, the invention also provides an assay method for identifying whether a patient, preferably a child, has a predisposition for developing type 1 diabetes and/or pancreatitis later in life comprising a step of detecting in a patient sample the presence or absence of (i) an influenza A virus or an expression product thereof, and/or (ii) an immune response against an influenza A virus. In some embodiments, the detection of (i) an influenza A virus or an expression product thereof, and/or (ii) an immune response against an influenza A virus in the patient sample indicates that s/he is predisposed to develop type 1 diabetes and/or pancreatitis later in life, particularly where the patient is already exhibiting pre-diabetic symptoms, e.g., insulitis. In other embodiments, absence of (i) an influenza A virus or an expression product thereof, and/or (ii) an immune response against an influenza A virus in the patient sample indicates that the patient has not been infected with influenza A virus. Such flu-negative patients are ideal candidates for treatment with composition(s) of the invention. Typically, such patients are young children, e.g., below the age of 5 years.

In some embodiments, the invention provides an assay method for prognosis of type 1 diabetes and/or pancreatitis comprising a step of detecting in a patient sample the presence or absence of (i) an influenza A virus or an expression product thereof, and/or (ii) an immune response against an A influenza virus. Optionally, the assay method further comprises the steps of: (a) identifying the level of (i) an A influenza virus or an expression product thereof, and/or (ii) an immune response against an influenza A virus in the patient sample; (b) comparing the level in the patient sample with a reference level; wherein: (i) a higher level in the patient sample indicates a poor prognosis; (ii) a lower level in the patient sample indicates a better prognosis

In some embodiments, the sample is a blood sample or a tracheal swab.

In some embodiments, the assay method is for use in a screening process, e.g., pediatric screening. For example, identification of children who test negative for (i) an influenza A virus or an expression product thereof, and/or (ii) an immune response against an influenza A virus in the patient sample indicates that the patient has not yet been infected with influenza A virus, and so is an ideal candidate for treatment with composition(s) of the invention.

In some embodiments, the patient is aged 70 years or less, and preferably between 0-15 years of age.

Any influenza A virus may be used in diagnostic, prognostic and/or prophylactic methods of the invention. Influenza A viruses suitable for use in diagnostic, prognostic and/or prophylactic methods of the invention may have any haemagglutinin type, e.g., H1, H2, H3, H4, H5, H6, H7, H8, H9, H10, H11, H12, H13, H14, H15 or H16, and any neuraminidas type, e.g., N1, N2, N3, N4, N5, N6, N7, N8 or N9.

Influenza virus strains for use with the invention can change from season to season, and may be pandemic or non-pandemic, In the current inter-pandemic period, vaccines typically include antigen(s) from two influenza A strains (H1N1 and H3N2) and one influenza B strain, and trivalent vaccines are typical. The invention may use antigen(s) from pandemic viral strains (i.e., strains to which the patient and the general human population are immunologically naive, in particular of influenza A virus), such as H2, H5, H7 or H9 subtype strains, and influenza vaccines for pandemic strains may be monovalent or may be based on a normal trivalent vaccine supplemented by a pandemic strain. Depending on which influenza virus strain is circulating and on the nature of the antigen, the invention may use one or more of HA subtypes H1, H2, H3, H4, H5, H6, H7, H8, H9, H10, H11, H12, H13, H14, H15 or H16. The invention may use one or more of influenza A virus NA subtypes N1, N2, N3, N4, N5, N6, N7, N8 or N9.

The characteristics of an influenza strain that give it the potential to cause a pandemic outbreak are: (a) it contains a new hemagglutinin compared to the hemagglutinins in currently-circulating human strains, i.e., one that has not been evident in the human population for over a decade (e.g., H2), or has not previously been seen at all in the human population (e.g., H5, H6 or H9, that have generally been found only in bird populations), such that the human population will be immunologically naive to the strain's hemagglutinin; (b) it is capable of being transmitted horizontally in the human population; and (c) it is pathogenic to humans. A virus with H5 hemagglutinin type is preferred for immunizing against pandemic influenza, such as a H5N1 strain. Other possible strains include H5N3, H9N2, H2N2, H7N1 and H7N7, and any other emerging potentially pandemic strains.

Preferably, the influenza A virus is H1N1, H2N2, H3N2, H5N1, H7N7, H1N2, H9N2, H7N2, H7N3 or H10N7; more preferably the influenza A virus is H1N1 or H3N2. Preferably, the influenza A virus is a non-systemic influenza A virus. Most preferably, the influenza A virus is H1N1, H3N2, H2N2.

Other strains whose antigens can usefully be included are strains which are resistant to antiviral therapy (e.g., resistant to oseltamivir [6] and/or zanamivir), including resistant pandemic strains [7].

### Administration of Antiviral Compounds

The invention provides a method for preventing or treating type 1 diabetes and/or pancreatitis in a patient, comprising a step of administering to the patient an antiviral compound effective against an A influenza virus. In some embodiments, antiviral compound(s) are administered to a patient who has been infected by A influenza virus. In preferred embodiments, antiviral compound(s) are administered to a patient who has not been infected by A influenza virus. Methods of determining whether a patient has been previously infected by influenza A virus are well known in the art, for example by detecting the presence of anti-influenza A virus antibodies in a patient sample, by ELISA.

In some embodiments, antiviral compound(s) are administered to a patient who is symptomatic of influenza A virus infection, or who has recently been symptomatic of influenza A virus infection, but is asymptomatic at the time of administration (e.g., 1, 2, 3, 4, 5, 6, 7, X, 9, 10, 11, 12, 13, 14, etc. days after symptoms have subsided). In such cases, administration of antiviral compound(s) typically decreases the duration and/or severity of influenza infection and symptoms. In view of the causal link between influenza A virus infection and type 1 diabetes, demonstrated by the inventors, antiviral treatment of influenza A virus infection will, m some cases, act as a prophylaxis for type 1 diabetes or as treatment for type 1 diabetes.

Various antiviral compounds effective against influenza viruses are known in the art, such as oseltamivir and/or zanamivir. These antivirals include, for example, neuraminidase inhibitors, such as a (3R,4R,5S)-4-acetylamino-5-amino-3 (1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g., the ethyl esters) and salts thereof (e.g., the phosphate salts). A preferred antiviral is (3R,4R,5S)-4-acetylamino-5-amino-3(1- ethylpropoxy)-1-cyclohexene-l-carboxylic acid, ethyl ester, phosphate (1:1), also known as oseltamivir phosphate (TAMIFLU). Another preferred antiviral is (2R,3R,4S)-4-guanidino-3-(prop-1-cn-2-ylamino)-2-((1R,2R)-1,2,3-trihydroxypropyl)-3 ,4-dihydro-2H-pyran-6-carboxylic acid, also known as zanamivir (RELENZA). Tamiflu has received FDA approval for prophylaxis of influenza A and B virus in patients aged 1 year and older. Relenza has received FDA approval for prophylaxis of influenza A and B virus in patients aged 5 years and older. Thus, when a patient is aged between 1 and 5 years, Tamiflu is the preferred antiviral. When a patient is aged 5 years or above, then Tamiflu and/or Relenza are preferred. Tamiflu and Relenza have also received FDA approval for treatment of uncomplicated acute illness due to influenza A or B virus infection in patients aged 1 year and older, and 7 years and older, respectively, when the patient has been symptomatic for no more than two days. Thus, when a symptomatic patient is aged between 1 and 7 years, Tamiflu is the preferred antiviral. When a symptomatic patient is aged 7 years or above, then Tamiflu and/or Relenza are preferred. Amantadine hydrochloride (SYMMETREL) had received pediatric approval for pediatric patients aged between 1-12 years. These and other antivirals may be used.

Further antivirals that may be useful with the invention include, but are not limited to: galangin (3,5,7-trihydroxyflavone); bupleurum kaoi; neopterin; Ardisia chinensis extract; galloyltricetifavans, such as 7-O-galloyltricetifavan and 7,4′-di-O-galloyltricetifavan; purine and pyrimidine cis-substituted cyclohexenyl and cyclohexanyl nucleosides; benzimidazole derivatives; pyridazinyl oxime ethers; enviroxime; disoxaril; arildone; PTU-23; HBB; S-7; 2-(3 ,4-dichloro-phenoxy)-5-nitrobenzonitrile; 6-bromo-2,3-disubstituted-4(3H)-quinazolinones; 3-methylthio-5-aryl-4-isothiazolecarbonitriles; quassinoids, such as simalikalactone D; 5′-Nor carbocyclic 5′-deoxy-5′-(isobutylthio)adenosine and its 2′,3′-dideoxy-2′,3′-didehydro derivative; oxathiin carboxanilide analogues; vinylacetylene analogs of enviroxime; Dehydroepiandrosterone (5-androsten-3 beta-ol-17-one, DHEA); flavans, isoflavans and isoflavenes substituted with chloro, cyano or amidino groups, such as substituted 3-(2H)-isoflavenes carrying a double bond in the oxygenated ring, e.g., 4′-chloro-6-cyanoflavan and 6-chloro-4′-cyanoflavan; 4-diazo-5-alkylsulphonamidopyrazoles; 3′-deoxy-3′-fluoro- and 2′-azido-3′-fluoro-2′,3′-dideoxy-D-ribofuranosides of natural heterocyclic bases; etc.

Mixtures of two or more antivirals may be used. For instance, reference 8 reports that certain combinations may show synergistic activity.

In addition to small organic antivirals, cytokine therapy may be used, e.g., with interferons. Compounds that elicit an interferon a response can also be used, e.g., inosine-containing nucleic acids such as ampligen.

Nucleic acid approaches can also be used against influenza virus, such as antisense or small inhibitory RNAs, to regulate virus production post-transcriptionally. Reference 9 demonstrates in vivo antiviral activity of antisense compounds administered intravenously to mice in experimental respiratory tract infections induced with influenza A virus. Type 1 diabetes may be treated or prevented by administering to a patient a nucleic acid, such as antisense or small inhibitory RNAs, specific to influenza A virus nucleic acid sequence(s). Such nucleic acids may be administered, e.g., as free nucleic acids, encapsulated nucleic acids (e.g., liposomally encapsulated), etc.

Immunisation

The invention provides a method for preventing or treating type 1 diabetes and/or pancreatitis in a patient, comprising a step of administering to the patient an immunogenic composition. The immunogenic composition includes an influenza A virus immunogen. Preferably, the immunogenic composition comprises an influenza A virus immunogen. Most preferably, the immunogenic composition comprises a non-systemic influenza A virus immunogen. Vaccines of the invention may be administered to patients at substantially the same time as (e.g., during the same medical consultation or visit to a healthcare professional) an antiviral compound, and in particular an antiviral compound active against influenza virus.

Influenza vaccines currently in general use are described in chapters 17 & 18 of reference 10. They are based on live virus or inactivated virus, and inactivated vaccines can be based on whole virus, ‘split’ virus or on purified surface antigens (including haemagglutinin and neuraminidase).

The invention uses an influenza A virus antigen, typically comprising hemagglutinin, to immunize a patient, preferably a child. The antigen will typically be prepared from influenza virions but, as an alternative, antigens such as haemagglutinin can be expressed in a recombinant host (e.g., in an insect cell line using a baculovirus vector) and used in purified form [11,12]. In general, however, antigens will be from virions.

The antigen may take the form of an inactivated virus or a live virus. Chemical means for inactivating a virus include treatment with an effective amount of one or more of the following agents: detergents, formaldehyde, formalin, β-propiolactone, or UV light. Additional chemical means for inactivation include treatment with methylene blue, psoralen, carboxyfullerene (C60) or a combination of any thereof. Other methods of viral inactivation are known in the art, such as for example binary ethylamine, acetyl ethyleneimine, or gamma irradiation. The INFLEXAL™ product is a whole virion inactivated vaccine.

Where an inactivated virus is used, the vaccine may comprise whole virion, split virion, or purified surface antigens (including hemagglutinin and, usually, also including neuraminidase).

An inactivated but non-whole cell vaccine (e.g., a split virus vaccine or a purified surface antigen vaccine) may include matrix protein, in order to benefit from the additional T cell epitopes that are located within this antigen. Thus a non-whole cell vaccine (particularly a split vaccine) that includes haemagglutinin and neuraminidase may additionally include M1 and/or M2 matrix protein. Useful matrix fragments are disclosed in reference 13. Nucleoprotein may also be present.

Virions can be harvested from virus-containing fluids by various methods. For example, a purification process may involve zonal centrifugation using a linear sucrose gradient solution that includes detergent to disrupt the virions. Antigens may then be purified, after optional dilution, by diafiltration.

Split virions are obtained by treating purified virions with detergents and/or solvents to produce subvirion preparations, including the ‘Tween-ether’ splitting process. Methods of splitting influenza viruses are well known in the art, e.g., see refs. 14-19, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g., cationic) surfactants. Suitable splitting agents include, but are not limited to: ethyl ether, polysorbate 80, deoxycholate, tri-N-butyl phosphate, alkylglycosides, alkylthioglycosides, acyl sugars, sulphobetaines, betaines, polyoxyethylenealkylethers, N,N-dialkyl-Glucamides, Hecameg, alkylphenoxy-polyethoxyethanols, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl-or nonylphenoxy polyoxyethanols (e.g., the Triton surfactants, such as Triton X-100 or Triton N101), nonoxynol 9 (NP9) Sympatens-NP/090,) polyoxyethylene sorbitan esters (the Tween surfactants), polyoxyethylene ethers, polyoxyethlene esters, etc. One useful splitting procedure uses the consecutive effects of sodium deoxycholate and formaldehyde, and splitting can take place during initial virion purification (e.g., in a sucrose density gradient solution). Thus a splitting process can involve clarification of the virion-containing material (to remove non-virion material), concentration of the harvested virions (e.g., using an adsorption method, such as CaHPO₄ adsorption), separation of whole virions from non-virion material, splitting of virions using a splitting agent in a density gradient centrifugation step (e.g., using a sucrose gradient that contains a splitting agent such as sodium deoxycholate), and then filtration (e.g., ultrafiltration) to remove undesired materials. Split virions can usefully be resuspended in sodium phosphate-buffered isotonic sodium chloride solution. The BEGRIVAC™, FLUARIX™, FLUZONE™ and FLUSHIELD™ products are split vaccines.

Purified surface antigen vaccines comprise the influenza surface antigens haemagglutinin and, typically, also neuraminidase. Processes for preparing these proteins in purified form are well known in the art. The FLUVIRIN™, AGRIPPAL™ and INFLUVAC™ products are subunit vaccines.

Another form of inactivated influenza antigen is the virosome [20] (nucleic acid free viral-like liposomal particles). Virosomes can be prepared by solubilization of influenza virus with a detergent followed by removal of the nucleocapsid and reconstitution of the membrane containing the viral glycoproteins. An alternative method for preparing virosomes involves adding viral membrane glycoproteins to excess amounts of phospholipids, to give liposomes with viral proteins in their membrane. The INFLEXAL V™ and INV A V AC™ products use virosomes.

The influenza antigen can be a live attenuated influenza virus (LAIV). LAIV vaccines can be administered by nasal spray and typically contain between 10⁶⁵ and 10⁷⁵ FFU (fluorescent focus units) of live attenuated virus per strain per dose. A LAIV strain can be cold-adapted (“ca”), i.e., it can replicate efficiently at 25° C., a temperature that is restrictive for replication of many wildtype influenza viruses. It may be temperature-sensitive (“ts”), i.e., its replication is restricted at temperatures at which many wild-type influenza viruses grow efficiently (37-39° C.). It may be attenuated (“att”), e.g., so as not to produce influenza-like illness in a ferret model of human influenza infection. The cumulative effect of the antigenic properties and the ca, ts, and att phenotype is that the virus in the attenuated vaccine can replicate in the nasopharynx to induce protective immunity in a typical human patient but does not cause disease, i.e., it is safe for general administration to the target human population. FL UMIST™ is a LAIV vaccine.

HA is the main immunogen in current inactivated influenza vaccines, and vaccine doses are standardised by reference to HA levels, typically measured by SRID. Existing vaccines typically contain about 15 μg of HA per strain, although lower doses can be used, e.g., for children, or in pandemic situations, or when using an adjuvant. Fractional doses such as ½ (i.e., 7.5 μg HA per strain), ¼ and ⅛ have been used, as have higher doses (e.g., 3× or 9× doses [21, 22]). Thus vaccines may include between 0.1 and 150 μg of HA per influenza strain, preferably between 0.1 and 50 μg, e.g., 0.1-20 μg, 0.1-15 μg, 0.1-10 μg, 0.1-7.5 μg, 0.5-5 μg, etc. Particular doses include, e.g., about 45, about 30, about 15, about 10, about 7.5, about 5, about 3.8, about 1.9, about 1.5, etc. per strain. A dose of 7.5 μg per strain is ideal for use in children.

For live vaccines, dosing is measured by median tissue culture infectious dose (TCID₅₀) rather than HA content, and a TCID₅₀ of between 10⁶ and 10⁸ (preferably between 10^{6.5}-10^{7.5}) per strain is typical.

Influenza virus strains for use in vaccines change from season to season. In the current inter-pandemic period, vaccines typically include two influenza A strains (H1N1 and H3N2) and one influenza B strain, and trivalent vaccines are typical for use with the invention. Preferably, compositions of the invention comprise antigen from an influenza A virus. Optionally compositions of the invention comprise antigen from an influenza B virus. Where the composition of the invention comprises antigen from influenza A virus(es), the invention may use seasonal and/or pandemic strains. Depending on the season and on the nature of the antigen included in the vaccine, the invention may include (and protect against) one or more of influenza A virus hemagglutinin subtypes H1, H2, H3, H4, H5, H6, H7, H8, H9, H10, H11, H12, H13, H14, H15 or H16. The vaccine may additionally include neuraminidase from any of NA subtypes N1, N2, N3, N4, N5, N6, N7, N8 or N9.

In some embodiments, compositions of the invention comprise immunogen(s) from pandemic influenza A virus strains. Characteristics of a pandemic strain are: (a) it contains a new hemagglutinin compared to the hemagglutinins in currently-circulating human strains, i.e., one that has not been evident in the human population for over a decade (e.g., H2), or has not previously been seen at all in the human population (e.g., H5, H6 or H9, that have generally been found only in bird populations), such that the vaccine recipient and the general human population are immunologically naive to the strain's hemagglutinin; (b) it is capable of being transmitted horizontally in the human population; and (c) it is pathogenic to humans. Pandemic strains include, but are not limited to, H2, H5, H7 or H9 subtype strains, e.g., H5N1, H5N3, H9N2, H2N2, H7N1 and H7N7 strains. Within the H5 subtype, a virus may fall into a number of clades, e.g., clade 1 or clade 2. Six sub-clades of clade 2 have been identified with sub-clades 1, 2 and 3 having a distinct geographic distribution and are particularly relevant due to their implication in human infections.

In some embodiments, compositions of the invention comprise influenza B virus immunogen(s). Influenza B virus currently does not display different HA subtypes, but influenza B virus strains do fall into two distinct lineages. These lineages emerged in the late 1980s and have HAs which can be antigenically and/or genetically distinguished from each other [23]. Current influenza B virus strains are either B/Victoria/2/87-like or B/Yamagata/16/88-like. These strains are usually distinguished antigenically, but differences in amino acid sequences have also been described for distinguishing the two lineages, e.g., B/Yamagata/16/88-like strains often (but not always) have HA proteins with deletions at amino acid residue 164, numbered relative to the ‘Lee40’ HA sequence [24]. The invention can be used with antigens from a B virus of either lineage.

Where a vaccine includes more than one strain of influenza, the different strains are typically grown separately and are mixed after the viruses have been harvested and antigens have been prepared. Thus a manufacturing process of the invention may include the step of mixing antigens from more than one influenza strain.

An influenza virus used with the invention may be a reassortant strain, and may have been obtained by reverse genetics techniques. Reverse genetics techniques [e.g., 25-29] allow influenza viruses with desired genome segments to be prepared in vitro using plasmids. Typically, it involves expressing (a) DNA molecules that encode desired viral RNA molecules, e.g., from pol I promoters or bacteriophage RNA polymerase promoters, and (b) DNA molecules that encode viral proteins, e.g., from pol II promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA can be used [30-32], and these methods will also involve the use of plasmids to express all or some (e.g., just the PB1, PB2, PA and NP proteins) of the viral proteins, with up to 12 plasmids being used in some methods. To reduce the number of plasmids needed, a recent approach [33] combines a plurality of RNA polymerase I transcription cassettes (for viral RNA synthesis) on the same plasmid (e.g., sequences encoding 1, 2, 3, 4, 5, 6, 7 or all 8 influenza A vRNA segments), and a plurality of protein-coding regions with RNA polymerase II promoters on another plasmid (e.g., sequences encoding 1, 2, 3, 4, 5, 6, 7 or all 8 influenza A mRNA transcripts). Preferred aspects of the reference 33 method involve: (a) PB1, PB2 and PA mRNA-encoding regions on a single plasmid; and (b) all 8 vRNA-encoding segments on a single plasmid. Including the NA and HA segments on one plasmid and the six other segments on another plasmid can also facilitate matters.

As an alternative to using poll promoters to encode the viral RNA segments, it is possible to use bacteriophage polymerase promoters [34]. For instance, promoters for the SP6, T3 or T7 polymerases can conveniently be used. Because of the species-specificity of pol I promoters, bacteriophage polymerase promoters can be more convenient for many cell types (e.g., MDCK), although a cell must also be transfected with a plasmid encoding the exogenous polymerase enzyme.

In other techniques it is possible to use dual pol I and pol II promoters to simultaneously code for the viral RNAs and for expressible mRNAs from a single template [35, 36].

Thus an influenza A virus may include one or more RNA segments from a A/PR/8/34 virus (typically 6 segments from A/PR/8/34, with the HA and N segments being from a vaccine strain, i.e., a 6:2 reassortant). It may also include one or more RNA segments from a A/WSN/33 virus, or from any other virus strain useful for generating reassortant viruses for vaccine preparation. An influenza A virus may include fewer than 6 (i.e., 0, 1, 2, 3, 4 or 5) viral segments from an AA/6/60 influenza virus (A/Ann Arbor/6/60). An influenza B virus may include fewer than 6 (i.e., 0, 1, 2, 3, 4 or 5) viral segments from an AA/1/66 influenza virus (B/Ann Arbor/1/66). Typically, the invention protects against a strain that is capable of human-to-human transmission, and so the strain's genome will usually include at least one RNA segment that originated in a mammalian (e.g., in a human) influenza virus. It may include NS segment that originated in an avian influenza virus.

Strains whose antigens can be included in the compositions may be resistant to antiviral therapy (e.g., resistant to oseltamivir [37] and/or zanamivir), including resistant pandemic strains [38].

HA used with the invention may be a natural HAas found in a virus, or may have been modified. For instance, it is known to modify HA to remove determinants (e.g., hyper-basic regions around the cleavage site between HA1 and HA2) that cause a virus to be highly pathogenic in avian species, as these determinants can otherwise prevent a virus from being grown in eggs.

The viruses used as the source of the antigens can be grown either on eggs (e.g., specific pathogen free eggs) or on cell culture. The current standard method for influenza virus growth uses embryonated hen eggs, with virus being purified from the egg contents (allantoic fluid). More recently, however, viruses have been grown in animal cell culture and, for reasons of speed and patient allergies, this growth method is preferred.

The cell line will typically be of mammalian origin. Suitable mammalian cells of origin include, but are not limited to, hamster, cattle, primate (including humans and monkeys) and dog cells, although the use of primate cells is not preferred. Various cell types may be used, such as kidney cells, fibroblasts, retinal cells, lung cells, etc. Examples of suitable hamster cells are the cell lines having the names BHK21 or HKCC. Suitable monkey cells are, e.g., African green monkey cells, such as kidney cells as in the Vero cell line [39-41]. Suitable dog cells are, e.g., kidney cells, as in the CLDK and MDCK cell lines.

Thus suitable cell lines include, but are not limited to: MDCK; CHO; CLDK; HKCC; 293T; BHK; Vero; MRC-5; PER.C6 [42]; FRhL2; WI-38; etc. Suitable cell lines are widely available, e.g., from the American Type Cell Culture (ATCC) collection [43], from the Coriell Cell Repositories [44], or from the European Collection of Cell Cultures (ECACC). For example, the ATCC supplies various different Vero cells under catalog numbers CCL-81, CCL-81.2, CRL-1586 and CRL-1587, and it supplies MDCK cells under catalog number CCL-34. PER.C6 is available from the ECACC under deposit number 96022940.

The most preferred cell lines are those with mammalian-type glycosylation. As a less-preferred alternative to mammalian cell lines, virus can be grown on avian cell lines [e.g., refs. 45-47], including cell lines derived from ducks (e.g., duck retina) or hens. Examples of avian cell lines include avian embryonic stem cells [45,48] and duck retina cells [46]. Suitable avian embryonic stem cells, include the EBx cell line derived from chicken embryonic stem cells, EB45, EB14, and EB14-074 [49]. Chicken embryo fibroblasts (CEF) may also be used. Rather than using avian cells, however, the use of mammalian cells means that vaccines can be free from avian DNA and egg proteins (such as ovalbumin and ovomucoid), thereby reducing allergenicity.

The most preferred cell lines for growing influenza viruses are MDCK cell lines [50-53], derived from Madin Darby canine kidney. The original MDCK cell line is available from the ATCC as CCL-34, but derivatives of this cell line may also be used. For instance, reference 50 discloses a MDCK cell line that was adapted for growth in suspension culture (‘MDCK 33016’, deposited as DSM ACC 2219). Similarly, reference 54 discloses a MDCK-derived cell line that grows in suspension in serum-free culture (‘B-702’, deposited as PERM BP-7449). Reference 55 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (PTA-6503). Reference 56 discloses MDCK cell lines with high susceptibility to infection, including ‘MDCK.5F1’ cells (ATCC CRL-12042). Any of these MDCK cell lines can be used.

Virus may be grown on cells in adherent culture or in suspension. Microcarrier cultures can also be used. In some embodiments, the cells may thus be adapted for growth in suspension.

Cell lines are preferably grown in serum-free culture media and/or protein free media. A medium is referred to as a serum-free medium in the context of the present invention in which there are no additives from serum of human or animal origin. The cells growing in such cultures naturally contain proteins themselves, but a protein-free medium is understood to mean one in which multiplication of the cells occurs with exclusion of proteins, growth factors, other protein additives and non-serum proteins, but can optionally include proteins such as trypsin or other proteases that may be necessary for viral growth.

Cell lines supporting influenza virus replication are preferably grown below 37° C. [57] (e.g., 30-36° C., or at about 30° C., 31° C., 32° C., 33° C., 34° C., 35° C., 36° C.) during viral replication.

Methods for propagating influenza virus in cultured cells generally includes the steps of inoculating a culture of cells with an inoculum of the strain to be grown, cultivating the infected cells for a desired time period for virus propagation, such as for example as determined by virus titer or antigen expression (e.g., between 24 and 168 hours after inoculation) and collecting the propagated virus. The cultured cells are inoculated with a virus (measured by PFU or TCTD50) to cell ratio of 1:500 to 1:1, preferably 1:100 to 1:5, more preferably 1:50 to 1:10. The virus is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g., monolayers) may be removed either by freeze-thawing or by enzymatic action to increase the viral content of the harvested culture supernatants. The harvested fluids are then either inactivated or stored frozen. Cultured cells may be infected at a multiplicity of infection (“m.o.i.”) of about 0.0001 to 10, preferably 0.002 to 5, more preferably to 0.001 to 2. Still more preferably, the cells are infected at a m.o.i of about 0.01. Infected cells may be harvested 30 to 60 hours post infection. Preferably, the cells are harvested 34 to 48 hours post infection. Still more preferably, the cells are harvested 38 to 40 hours post infection. Protcases (typically trypsin) are generally added during cell culture to allow viral release, and the proteases can be added at any suitable stage during the culture, e.g., before inoculation, at the same time as inoculation, or after inoculation [57].

In preferred embodiments, particularly with MDCK cells, a cell line is not passaged from the master working cell bank beyond 40 population-doubling levels.

The viral inoculum and the viral culture are preferably free from (i.e., will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses [58]. Absence of herpes simplex viruses is particularly preferred.

Where virus has been grown on a cell line then it is standard practice to minimize the amount of residual cell line DNA in the final vaccine, in order to minimize any oncogenic activity of the DNA.

Thus a vaccine composition prepared according to the invention preferably contains less than 10 ng (preferably less than 1 ng, and more preferably less than 100 pg) of residual host cell DNA per dose, although trace amounts of host cell DNA may be present.

Vaccines containing <10 ng (e.g., <1 ng, <100 pg) host cell DNA per 15 μg of haemagglutinin are preferred, as are vaccines containing <10 ng (e.g., <1 ng, <100 pg) host cell DNA per 0.25 ml volume. Vaccines containing <10ng (e.g., <1 ng, <100 pg) host cell DNA per 50 μg of haemagglutinin are more preferred, as are vaccines containing <10 ng (e.g., <1 ng, <100 pg) host cell DNA per 0.5 ml volume.

It is preferred that the average length of any residual host cell DNA is less than 500 bp, e.g., less than 400 bp, less than 300 bp, less than 200 bp, less than 100 bp, etc.

Contaminating DNA can be removed during vaccine preparation using standard purification procedures, e.g., chromatography, etc. Removal of residual host cell DNA can be enhanced by nuclease treatment, e.g., by using a DNase. A convenient method for reducing host cell DNA contamination is disclosed in references 59 & 60, involving a two-step treatment, first using a DNase (e.g., Benzonase), which may be used during viral growth, and then a cationic detergent (e.g., CTAB), which may be used during virion disruption. Removal by β-propiolactone treatment can also be used.

Measurement of residual host cell DNA is now a routine regulatory requirement for biologicals and is within the normal capabilities of the skilled person. The assay used to measure DNA will typically be a validated assay [61,62]. The performance characteristics of a validated assay can be described in mathematical and quantifiable terms, and its possible sources of error will have been identified. The assay will generally have been tested for characteristics such as accuracy, precision, specificity. Once an assay has been calibrated (e.g., against known standard quantities of host cell DNA) and tested then quantitative DNA measurements can be routinely performed. Three main techniques for DNA quantification can be used: hybridization methods, such as Southern blots or slot blots [63]; immunoassay methods, such as the Threshold™ System [64]; and quantitative PCR [65]. These methods are all familiar to the skilled person, although the precise characteristics of each method may depend on the host cell in question, e.g., the choice of probes for hybridization, the choice of primers and/or probes for amplification, etc. The Threshold™ system from Molecular Devices is a quantitative assay for picogram levels of total DNA, and has been used for monitoring levels of contaminating DNA in biopharmaceuticals [64]. A typical assay involves non-sequence-specific formation of a reaction complex between a biotinylated ssDNA binding protein, a urease-conjugated anti-ssDNA antibody, and DNA. All assay components are included in the complete Total DNA Assay Kit available from the manufacturer. Various commercial manufacturers offer quantitative PCR assays for detecting residual host cell DNA, e.g., AppTec™ Laboratory Services, BioReliance™, Althea Technologies, etc. A comparison of a chemiluminescent hybridisation assay and the total DNA Threshold™ system for measuring host cell DNA contamination of a human viral vaccine can be found in reference 66. The influenza virus immunogen may take various forms.

As an alternative to delivering polypeptide-based immunogens themselves, nucleic acids encoding the polypeptides may be administered such that, after delivery to the body, the polypeptides are expressed in situ. Nucleic acid immunization typically utilizes a vector, such as a plasmid, comprising: (i) a promoter; (ii) a sequence encoding the immunogen, operably linked to said promoter; and (iii) a selectable marker. Vectors often further comprise (iv) an origin of replication; and (v) a transcription terminator downstream of and operably linked to (ii). Components (i) & (v) will usually be eukaryotic, whereas (iii) and (iv) are prokaryotic.

A polypeptide used in an immunogenic composition may have an amino acid sequence of a natural influenza polypeptide (precursor or mature form) or it may be artificial, e.g., it may be a fusion protein or it may comprise a fragment (e.g., including an epitope) of a natural influenza sequence.

### Adjuvants

Vaccines and compositions of the invention may advantageously include an adjuvant, which can function to enhance the immune responses (humoral and/or cellular) elicited in a patient who receives the composition. The use of adjuvants with influenza vaccines has been described before. In U.S. Pat. No. 6,372,223 and in WO00/15251, aluminum hydroxide was used, and in WO01/22992, a mixture of aluminum hydroxide and aluminum phosphate was used. Hehme et al. (2004) Virus Res. 103(1-2):163-71 also described the use of aluminum salt adjuvants. The FLUAD™ product from Novartis Vaccines includes an oil-in-water emulsion. Adjuvant-active substances are discussed in more detail in Vaccine Design: The Subunit and Adjuvant Approach (eds. Powell & Newman) Plenum Press 1995 [ISBN 0-306-44867-X], and in Vaccine Adjuvants: Preparation Methods and Research Protocols (Volume 42 of Methods in Molecular Medicine series) Ed. O'Hagan [ISBN: 1-59259-083-7].

Adjuvants that can be used with the invention include, but are not limited to, those described in WO2008/068631. Compositions may include two or more of said adjuvants. Antigens and adjuvants in a composition will typically be in admixture.

### Oil-in-Water Emulsion Adjuvants

Oil-in-water emulsions are preferred adjuvants for use with the invention as they have been found to be particularly suitable for use in adjuvanting influenza virus vaccines. Various such emulsions are known, and they typically include at least one oil and at least one surfactant, with the oil(s) and surfactant(s) being biodegradable (metabolisable) and biocompatible. The oil droplets in the emulsion are generally less than 5 μm in diameter, and advantageously the emulsion comprises oil droplets with a sub-micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used, e.g., obtained from the jojoba bean. Seed oils include safflower oil, cottonseed oil, sunflower seed oil, sesame seed oil, etc. In the grain group, com oil is the most readily available, but the oil of other cereal grains such as wheat, oats, rye, rice, teff, triticale, etc. may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEP AL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used, e.g., Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ (WO90/14837; Podda & Del Giudice (2003) Expert Rev Vaccines 2:197-203; Podda (2001) Vaccine 19: 2673-2680), as described in more detail in Chapter 10 of Vaccine Design: The Subunit and Adjuvant Approach (eds. Powell & Newman) Plenum Press 1995 [ISBN 0-306-44867-X], and in chapter 12 of Vaccine Adjuvants: Preparation Methods and Research Protocols (Volume 42 of Methods in Molecular Medicine series) Ed. O'Hagan [ISBN: 1-59259-083-7]. The MF59 emulsion advantageously includes citrate ions, e.g., 10 mM sodium citrate buffer.
An emulsion of squalene, a tocopherol, and polysorbate 80. The emulsion may include phosphate buffered saline. It may also include Span 85 (e.g., at 1%) and/or lecithin. These emulsions may have from 2 to 10% squalene, from 2 to 10% tocopherol and from 0.3 to 3% polysorbate 80, and the weight ratio of squalene:tocopherol is preferably <1 as this provides a more stable emulsion. Squalene and polysorbate 80 may be present volume ratio of about 5:2 or at a weight ratio of about 11:5. Thus the three components (squalene, tocopherol, polysorbate XO) may be present at a weight ratio of 1068:1186:485 or around 55:61:25. One such emulsion (‘AS03’) can be made by dissolving Tween 80 in PBS to give a 2% solution, then mixing 90 ml of this solution with a mixture of (5g of DL-α-tocopherol and 5 ml squalene), then microfluidising the mixture. The resulting emulsion may have submicron oil droplets, e.g., with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 (WO2008/043774), e.g., in the ratios discussed above.
An emulsion of squalene, a tocopherol, and a Triton detergent (e.g., Triton X-100). The emulsion may also include a 3d-MPL (see below). The emulsion may contain a phosphate buffer.
An emulsion comprising a polysorbate (e.g., polysorbate 80), a Triton detergent (e.g.,

Triton X-100) and a tocopherol (e.g., an α-tocopherol succinate). The emulsion may include these three components at a mass ratio of about 75:11:10 (e.g., 750 μ/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalene, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant (Allison & Byars (1992) Res Immunol 143:519-25) (0.05-1% Thr-MDP, 5% squalane, 2.5% Pluronic L121 and 0.2% polysorbate 80). It can also be used without 20 the Thr-MDP, as in the “AF” adjuvant (Hariharan et al. (1995) Cancer Res 55:3486-9) (5% squalane, 1.251 Yo Pluronic L121 and 0.2% polysorbate XO). Microfluidisation is preferred.
An emulsion comprising squalene, an aqueous solvent, a polyoxyethylene alkyl ether hydrophilic nonionic surfactant (e.g., polyoxyethylene (12) cetostearyl ether) and a hydrophobic nonionic surfactant (e.g., a sorbitan ester or mannide ester, such as sorbitan monoleate or ‘Span 80’). The emulsion is preferably thermoreversible and/or has at least 90% of the oil droplets (by volume) with a size less than 200 nm (U.S. 2007/014805). The emulsion may also include one or more of: alditol; a cryoprotective agent (e.g., a sugar, such as dodecylmaltoside and/or sucrose); and/or an alkylpolyglycoside. Such emulsions may be lyophilized. The emulsion may include squalene:polyoxyethylene cetostearyl ether:sorbitan oleate:mannitol at a mass ratio of 330:63:49:61.
An emulsion of squalene, poloxamer 105 and Abil-Care (Suli et al. (2004) Vaccine 22(25-26):3464-9). The final concentration (weight) of these components in adjuvanted vaccines are 5% squalene, 4% poloxamer 105 (pluronic polyol) and 2% Abil-Care 85 (BisPEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in WO95111700, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in U.S. Pat. No. 6,080,725, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion comprising a mineral oil, a non-ionic lipophilic ethoxylated fatty alcohol, and a non-ionic hydrophilic surfactant (e.g., an ethoxylated fatty alcohol and/or polyoxyethylene-polyoxypropylene block copolymer) (WO2006/113373).
An emulsion comprising a mineral oil, a non-ionic hydrophilic ethoxylated fatty alcohol, and a non-ionic lipophilic surfactant (e.g., an ethoxylated fatty alcohol and/or polyoxyethylene-polyoxypropylene block copolymer) (WO2006/113373).
An emulsion in which a saponin (e.g., QuilA or QS21) and a sterol (e.g., a cholesterol) are associated as helical micelles (WO2005/097181).

Antigens and adjuvants in a composition will typically be in admixture at the time of delivery to a patient. The emulsions may be mixed with antigen during manufacture, or extemporaneously, at the time of delivery. Thus the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g., between 5:1 and 1:5) but is generally about 1:1.

After the antigen and adjuvant have been mixed, haemagglutinin antigen will generally remain in aqueous solution but may distribute itself around the oil/water interface. In general, little if any haemagglutinin will enter the oil phase of the emulsion.

Where a composition includes a tocopherol, any of the α, β, γ, δ, ξ or tocopherols can be used, but α-tocopherols are preferred. The tocopherol can take several forms, e.g., different salts and/or isomers. Salts include organic salts, such as succinate, acetate, nicotinate, etc. D-α-tocopherol and DL-α-tocopherol can both be used. Tocopherols are advantageously included in vaccines for use in elderly patients (e.g., aged 60 years or older) because vitamin E has been reported to have a positive effect on the immune response in this patient group (Han et al. (2005) Impact of Vitamin E on Immune Function and Infectious Diseases in the Aged at Nutrition, Immune functions and Health EuroConference, Paris, 9-10 Jun. 2005). They also have antioxidant properties that may help to stabilize the emulsions (U.S. Pat. No. 6,630,161). A preferred α-tocopherol is DL-α-tocopherol, and the preferred salt of this tocopherol is the succinate. The succinate salt has been found to cooperate with TNF-related ligands in vivo. Moreover, α-tocopherol succinate is known to be compatible with influenza vaccines and to be a useful preservative as an alternative to mercurial compounds (WO02/097072).

As mentioned above, oil-in-water emulsions comprising squalene are particularly preferred. In some embodiments, the squalene concentration in a vaccine dose may be in the range of 5-15 mg (i.e., a concentration of 10-30 mg/ml, assuming a 0.5 ml dose volume). It is possible, though, to reduce the concentration of squalene (WO2007/052155; WO2008/128939), e.g., to include <5 mg per dose, or even <1.1 mg per dose. For example, a human dose may include 9.75 mg squalene per dose (as in the FLUAD™ product: 9.75 mg squalene, 1.175 mg polysorbate 80, 1.175 mg sorbitan trioleate, in a 0.5 ml dose volume), or it may include a fractional amount thereof, e.g., ¾, ⅔, ½, ⅓, ¼, ⅕, ⅙, 1/7, ⅛, 1/9, or 1/10. For example, a composition may include 7.31 mg squalene per dose (and thus 0.88 mg each of polysorbate 80 and sorbitan trioleate), 4.875 mg squalene/dose (and thus 0.588 mg each of polysorbate 80 and sorbitan trioleate), 3.25 mg squalene/dose, 2.438 mg/dose, 1.95 mg/dose, 0.975 mg/dose, etc. Any of these fractional dilutions of the FLUAD™-strength MF59 can be used with the invention.

As mentioned above, antigen/emulsion mixing may be performed extemporaneously, at the time of delivery. Thus the invention provides kits including the antigen and adjuvant components ready for mixing. The kit allows the adjuvant and the antigen to be kept separately until the time of use. The components are physically separate from each other within the kit, and this separation can be achieved in various ways. For instance, the two components may be in two separate containers, such as vials. The contents of the two vials can then be mixed, e.g., by removing the contents of one vial and adding them to the other vial, or by separately removing the contents of both vials and mixing them in a third container. In a preferred arrangement, one of the kit components is in a syringe and the other is in a container such as a vial. The syringe can be used (e.g., with a needle) to insert its contents into the second container for mixing, and the mixture can then be withdrawn into the syringe. The mixed contents of the syringe can then be administered to a patient, typically through a new sterile needle. Packing one component in a syringe eliminates the need for using a separate syringe for patient administration. In another preferred arrangement, the two kit components are held together but separately in the same syringe, e.g., a dual-chamber syringe, such as those disclosed in WO2005/089837; U.S. Pat. No. 6,692,468; WO00/07647; WO99/17820; U.S. Pat. No. 5,971,953; U.S. Pat. No. 4,060,082; EP-A-0520618; WO98/01174 etc. When the syringe is actuated (e.g., during administration to a patient) then the contents of the two chambers are mixed. This arrangement avoids the need for a separate mixing step at the time of use.

### NK Modulation

NK cells are a subset of lymphocytes that act as an initial immune defense against tumor cells and virally infected cells. There exists evidence that NK cell dysfunction plays a role in the development of type 1 diabetes (see, e.g., references 67 and 68). Inhibition of NK cells may thus have therapeutic potential in infected patients. Thus, the invention provides a method for preventing or treating type 1 diabetes in a patient, comprising administering an immunogenic composition and/or an antiviral of the invention and also an immunomodulatory compound effective to inhibit natural killer cell activity. In general, however, total inhibition is not desirable.

Compounds effective to inhibit NK function include, but are not limited to: steroids, such as methylprednisolone; tributyltin; Ly49 ligands, such as H-2D(d); soluble HLA-G1; CD94/NKG2A; CD244 ligands; etc.

Compounds may act directly or indirectly on the NK cells. For example, tributyltin acts directly on NK cells. In contrast, CD4+CD25+ T regulatory cells can inhibit NK cells, and so a compound may be administered to a patient in order to promote such CD4+CD25+ T cells and thereby indirectly inhibit NK cells.

Assays for Diagnosis and/or Prognosis

It will be appreciated that “diagnosis” in the context of this invention relates to the identification of a predisposition in a patient, e.g., a child, for developing type 1 diabetes and/or pancreatitis later in life, rather than a definite clinical diagnosis of type 1 diabetes and/or pancreatitis in a patient per se. Where a patient is identified as having a disposition for developing type 1 diabetes and/or pancreatitis later in life, symptoms of type 1 diabetes and/or pancreatitis typically occur at least 1 year after diagnosis, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more years after diagnosis. In some cases however, where patient is identified as having a disposition for developing type 1 diabetes and/or pancreatitis later in life, symptoms of type 1 diabetes and/or pancreatitis typically occur within 1 year, e.g., within 1 month, within 2 months, within 3 months, within 6 months, or within 9 months of diagnosis. Detection described herein may be performed in vivo or in vitro.

Symptoms of type 1 diabetes are well known and typically include feeling very thirsty, feeling hungry, feeling tired or fatigued, having blurry eyesight, losing the feeling in the feet or feeling a tingling sensation in the feet, losing weight without trying to do so, increased frequency of urination, deep breathing, rapid breathing, flushed face, fruity breath odor, nausea, vomiting, inability to keep down fluids, stomach pain, headache, nervousness, heart palpitations, sweating, shaking, and/or weakness etc. Symptoms of pancreatitis are also well known and typically include pain, particularly radiating from the front of the abdomen through to the back, nausea, fever and/or chills, swollen abdomen, rapid heartbeat, fatigue, feeling lightheaded, feeling feint, lethargy irritability, confusion, difficulty concentrating, headache, weight loss, bleeding, and/or jaundice etc.

Accordingly, the invention provides diagnostic assay methods comprising a step of detecting in a patient sample the presence or absence of (a) an influenza A virus or an expression product thereof, and/or (b) an immune response against an influenza A virus. Detection of a presence indicates that the patient has been infected by influenza A virus and is thus at risk of the downstream diabetes-related and/or pancreatitis-related consequences. Assays of the invention can therefore be used for determining whether a patient has an increased risk of developing type 1 diabetes later in life, i.e., for determining whether a patient (e.g., a child) has a predisposition for developing type 1 diabetes. Similarly, assays of the invention can be used for determining whether a patient has an increased risk of developing pancreatitis later in life, i.e., for determining whether a patient (e.g., a child) has a predisposition for developing pancreatitis. Thus, in one embodiment, detection of a presence of an influenza A virus or an expression product thereof, and/or an immune response against an influenza A virus indicates a predisposition for 20 developing type 1 diabetes and/or pancreatitis.

Detection of an absence of (i) an influenza A virus or an expression product thereof, and/or (i) an immune response against an influenza A virus in a patient sample, indicates that the patient (typically a child) has not yet been infected with influenza A virus. Such patients are preferred candidates for treatment with composition(s) of the invention.

The inventors found that influenza A virus infection is associated with pancreatic damage. The level of influenza A virus infection can therefore indicate prognosis of type 1 diabetes and/or pancreatitis. For example, higher level influenza A virus infection leads to more severe pancreatic damage and thus a more severe presentation of type 1 diabetes and/or pancreatitis. Typically, prognosis of type 1 diabetes and/or pancreatitis in a patient involves comparing the level(s) of an influenza A virus or an expression product thereof, and/or an immune response against an influenza A virus in the patient sample, with the level(s) in a reference level. The reference level is preferably a level observed another patient(s), for whom the severity of type 1 diabetes and/or pancreatitis has been determined.

Thus, in some embodiments, detection of a high level of an influenza A virus or an expression product thereof, and/or an immune response against an influenza A virus indicates a poor prognosis for type 1 diabetes and/or pancreatitis, e.g., compared to a reference level. Conversely, a low detected level of an A influenza virus or an expression product thereof, and/or an immune response against an influenza A virus in a patient sample indicates a better prognosis for type 1 diabetes and/or pancreatitis, e.g., compared to a reference level.

Assay methods of the invention can be used as part of a screening process, with positive samples being subjected to further analysis. In general, the invention will be used to detect influenza A virus infection, in particular in relation to pancreatic beta cells, and the presence of infection will be used, alone or in combination with other test results, as the basis of diagnosis or prognosis. Preferably, assay methods of the invention are for identifying whether a patient has a predisposition for developing type 1 diabetes and/or for determining prognosis.

Assay methods of the invention may detect an influenza virus (e.g., its single-stranded RNA genome, a provirion, a virion), an expression product of an influenza virus (e.g., its anti-genome, a viral mRNA transcript, an encoded polypeptide such as, for example, NS1, PB-1-F2, hemagglutinin, neuraminidase, matrix protein (M1 and/or M2), ribonucleoprotein, nucleoprotein, polymerase complex (PB1, PB2, PA) or subunits thereof, nuclear export protein etc.), or the product of an immune response against an influenza virus (e.g., an antibody against a viral polypeptide, a T cell recognizing a viral polypeptide).

A useful method for detecting RNA is the polymerase chain reaction, and in particular RT-PCR (reverse transcriptase PCR). Further details on nucleic acid amplification methods are given below.

Various techniques are available for detecting the presence or absence of polypeptides in a sample. These are generally immunoassay techniques which are based on the specific interaction between an antibody and an antigenic amino acid sequence in the polypeptide. Suitable techniques include standard immunohistological methods, ELISA, RIA, FIA, immunoprecipitation, immunofluorescence, etc. Sandwich assays are typical. Antibodies against various influenza viruses are already commercially available.

Polypeptides can also be detected by functional assays, e.g., assays to detect binding activity or enzymatic activity. Another way of detecting polypeptides of the invention is to use standard proteomics techniques, e.g., purify or separate polypeptides and then use peptide sequencing. For example, polypeptides can be separated using 2D-PAGE and polypeptide spots can be sequenced (e.g., by mass spectroscopy) in order to identify if a sequence is present in a target polypeptide. Some of these techniques may require the enrichment of target polypeptides prior to detection; other techniques may be used directly, without the need for such enrichment.

Antibodies raised against an influenza virus may be present in a sample and can be detected by conventional immunoassay techniques, e.g., using influenza virus polypeptides, which will typically be immobilized.

### Prevention and Therapy

The invention can be used to prevent type 1 diabetes and/or pancreatitis in a patient. Such patients will not already be suffering from type 1 diabetes and/or pancreatitis, but they will be at risk of developing type 1 diabetes and/or pancreatitis. Such patients may be exhibiting pre-diabetic symptoms, e.g., insulitis. Prevention encompasses both (i) reducing the risk that they will develop type 1 diabetes, and (ii) lengthening the time before they develop type 1 diabetes. Because it has been found that influenza A virus infection leads to pancreatitis, the invention can also be used to prevent or treat pancreatitis in pre-diabetic patients and/or pre-pancreatitis patients. Such treatment or prevention is a further way in which the development and onset of type 1 diabetes and/or pancreatitis can be prevented.

In some embodiments, the invention can also be used to treat type 1 diabetes and/or pancreatitis in a patient. For instance, therapeutic immunization or antiviral treatment may be used to clear an influenza virus infection and then beta cell regeneration can be permitted (optionally in combination with treatment of the autoimmune aspect of type 1 diabetes). The method may be combined with islet transplantation or the transplantation of beta cell precursors or stem cells. The terms “treatment”, “treating”, “treat” and the like refer to obtaining a desired pharmacologic and/or physiologic effect. The effect may be therapeutic in terms of a partial or complete stabilization or cure for type 1 diabetes and/or adverse effect attributable to type 1 diabetes. “Treatment” includes inhibiting a disease symptom (i.e., arresting its development) and relieving the disease symptom, (i.e., causing regression of the disease or symptom).

Therapeutic immunization or antiviral treatment as described above may be used to clear an influenza virus infection and then beta cell regeneration can be permitted (optionally in combination with treatment of the autoimmune aspect of type 1 diabetes) in a patient suffering from pre-diabetic symptom(s) (e.g., insulitis), and who is thus at higher risk for developing type 1 diabetes.

The invention can be used in conjunction with methods of type 1 diabetes prevention and/or treatment. Methods of treating type 1 diabetes include, for example, administration of cyclosporin A, administration of anti-CD3 antibodies, e.g., teplizumab and/or otelixizumab, administration of anti-CD20 antibodies, e.g., rituximab, insulin therapy, vaccination with GAD65 (an autoantigen involved in type 1 diabetes), pancreas transplantation, islet cell transplantation etc. There is at present no established method for preventing type 1 diabetes. However, there is thought to be a link between development of diabetes and intake of cow's milk as an infant (see reference 69), and so some doctors recommend breast feeding children who have parents or siblings with type 1 diabetes, and limiting the child's intake of cow's milk.

The invention can be used with a wide variety of patients, but some embodiments are more useful for specific patient groups. For instance, some embodiments will usually be applied only with patients having a definite influenza virus infection, whereas other embodiments may be focused on patients known to be at high risk of developing type 1 diabetes (e.g., with a familial history of the disease, with a HLA-DR3 haplotype and/or a HLA-DR4 haplotype, etc.). For instance, the administration of antiviral compounds will typically be used in pre-diabetic patients having a viral infection, whereas prophylactic immunization will be used more widely (e.g., in high risk groups such as children who test negative for (i) an influenza A virus or an expression product thereof, and/or (ii) an immune response against an influenza A virus in the patient sample, or in the population as a whole).

A preferred type of patient for use with diagnostic, prognostic and prophylactic methods of the invention is a patient who has insulitis but has not yet developed type 1 diabetes.

### The Patient

The inventors propose that IAV infection may affect the pancreas at any age, and so the patient may be of any age for prophylactic, diagnostic, treatment and/ or prognostic embodiments of the invention. Typically, the patient is 70 years old or less, e.g., 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years of age, or less.

Typically, the patient is at least 1 month old, e.g., 1 month, 3 months, 6 months, 9 months, and preferably at least 1 year old, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more years old. Preferably, the patient is at least 5 years of age. More preferably, the patient is at least 7 years of age. Most preferably, the patient is at least 12 years of age.

The inventors have demonstrated a link between influenza A virus infection and the development of pancreatitis and/or type 1 diabetes in a patient. The inventors thus propose that the frequency and/or severity of influenza A virus infection in a patient affects the risk of developing pancreatitis and/or type 1 diabetes later in life, and may also affect the symptom severity (i.e., high frequency and/or severe infection(s) likely cause increased risk of developing pancreatitis and/or type 1 diabetes later in life, and may also increase the symptom severity). Therefore, to minimize the risk of developing pancreatitis and/or type 1 diabetes later in life, and to minimize the symptom severity, the patient is preferably flu-naive, or has had minimal exposure to flu.

Therefore, for prophylactic embodiments of the invention in particular, the patient is preferably a child, because children have typically had lower exposure to influenza A virus infection than adults. For embodiments of the invention, the child is preferably aged between 0-15 years, e.g., 0-10, 5-15, 0-5 (e.g., 0-3 or 3-5), 5-10 (e.g., 5-7 or 7-10) or 10-15 (e.g., 10-13 or 13-15) years of age. Typically the child will be at least 6 months old, e.g., in the range 6-72 months old (inclusive) or in the range 6-36 months old (inclusive), or in the range 36-72 months old (inclusive). Children in these age ranges may in some embodiments be less than 30 months old, or less than 24 months old. For example, a composition may be administered to them at the age of 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months; or at 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or 71 months; or at 36 or 72 months. The child is preferably aged between 0 months and 72 months, and ideally between 0 months and 36 months. Thus, the child may be immunized before their 3rd or 6th birthday.

### Patient Samples

Various embodiments of the invention require samples that have been obtained from patients. These samples will generally comprise cells (e.g., pancreatic cells, including beta cells). These may be present in a sample of tissue (e.g., a biopsy), or may be cells which have escaped into circulation. In some embodiments, however, the sample will be cell-free, e.g., from a body fluid that may contain influenza virions in the absence of patient cells, or a purified cell-free blood sample that may contain anti-viral antibodies.

In general, therefore, the patient sample is tissue sample or a blood sample. In some embodiments, the sample is a tracheal swab. Other possible sources of patient samples include isolated cells, whole tissues, or bodily fluids (e.g., blood, plasma, serum, urine, pleural effusions, cerebro-spinal fluid, etc.).

Expression products may be detected in the patient sample itself, or may be detected in material derived from the sample (e.g., the lysate of a cell sample, the supernatant of such a cell lysate, a nucleic acid extract of a cell sample, DNA reverse transcribed from a RNA sample, polypeptides translated from a RNA sample, cells derived from culturing cells extracted from a patient, etc.). These derivatives are still “patient samples” within the meaning of the invention.

Assay methods of the invention can be conducted in vitro or in vivo.

In some embodiments of the invention a control may be used, against which influenza virus levels in a patient sample can be compared. Analysis of the control sample gives a baseline level against which a patient sample can be compared. A negative control may be a sample from an uninfected patient, or it may be material not derived from a patient, e.g., a buffer. A positive control will be a sample with a known level of analyte. Other suitable positive and negative controls will be apparent to the skilled person.

Analyte in the control can be assessed at the same time as in the patient sample. Alternatively, a patient sample can be assessed separately (earlier or later). Rather than actually compare two samples, however, the control may be an absolute value i.e., a level of analyte which has been empirically determined from previous samples (e.g., under standard conditions).

The invention provides an immunoassay method, comprising the step of contacting a patient sample with a polypeptide or antibody of the invention.

### Nucleic Acids

Nucleic acid sequences encoding influenza A viruses are known in the art, and may be used in compositions and/or methods of the invention. The invention also provides nucleic acid comprising the complement (including the reverse complement) of such nucleotide sequences for use in compositions and/or methods of the invention. Nucleic acids may be used in prevention or treatment embodiments of the invention, e.g., for antisense and/or for use in DNA-based influenza vaccine to prevent development of type 1 diabetes and/or pancreatitis later in a patient's life. Nucleic acids may also be used in detection methods of the invention, e.g., for probing, for use as primers, etc. for use in identifying influenza A virus RNA in a sample and determining whether a patient has a predisposition for developing type 1 diabetes and/or pancreatitis later in life.

The invention also provides nucleic acid encoding polypeptides of the invention, preferably proteolytic products of the influenza A virus polyprotein for use in compositions and/or methods of the invention.

Primers and probes of the invention, and other nucleic acids used for hybridization, are preferably between 10 and 30 nucleotides in length (e.g., 10, 11, 12, 13, 14, 15, 16, 17, 18, 19, 20, 21, 22, 23, 24, 25, 26, 27, 28, 29, or 30 nucleotides).

The invention provides a process for detecting influenza virus in a biological sample (e.g., blood), comprising the step of contacting nucleic acid according to the invention with the biological sample under hybridising conditions. The process may involve nucleic acid amplification (e.g., PCR, SDA, SSSR, LCR, TMA, NASBA, etc.) or hybridisation (e.g., microarrays, blots, hybridisation with a probe in solution, etc.). For example, the invention provides a process for detecting an influenza virus nucleic acid in a sample, comprising the steps of: (a) contacting a nucleic probe according to the invention with a biological sample under hybridising conditions to form duplexes; and (b) detecting said duplexes.

### Polypeptides

Polypeptide sequences encoding influenza A viruses are known in the art and may be used in compositions and/or methods of the invention. Preferably, polypeptide sequences for use with the invention comprise at least one T-cell or, preferably, a B-cell epitope of the sequence. T- and B-cell epitopes can be identified empirically (e.g., using PEPSCAN [70,71] or similar methods), or they can be predicted (e.g., using the Jameson-Wolf antigenic index [72], matrix-based approaches [73], TEPITOPE [74], neural networks [75], OptiMer & EpiMer [76, 77], ADEPT [78], Tsites [79], hydrophilicity [80], antigenic index [81] or the methods disclosed in reference 82 etc.). Such polypeptide(s) may be used in immunogenic compositions of the invention, e.g., for use in preventing or treating type 1 diabetes and/or pancreatitis in a patient. Such polypeptide(s) may also be used for diagnosis, e.g., for detecting anti-influenza A virus antibodies in a sample, and so determining whether a patient has a predisposition for developing type 1 diabetes and/or pancreatitis later in life.

Polypeptides of the invention are generally at least 7 amino acids in length (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 amino acids or longer).

For certain embodiments of the invention, polypeptides are preferably at most 500 amino acids in length (e.g., 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24 ,23, 22, 21, 20, 19, 18, 17, 16, 15 amino acids or shorter).

### Antibodies

The invention provides antibody that binds to a polypeptide of the invention for use in compositions and/or methods of the invention. In some embodiments, such antibodies are for preventing or treating type 1 diabetes and/or pancreatitis, e.g., by passive immunization against influenza A virus infection. In other embodiments, such antibodies are for methods of diagnosis, e.g., for detecting anti-influenza A virus in a sample, and so determining whether a patient has a predisposition for developing type 1 diabetes and/or pancreatitis later in life. Antibodies of the invention may be polyclonal or monoclonal.

Antibodies of the invention may include a label. The label may be detectable directly, such as a radioactive or fluorescent label. Alternatively, the label may be detectable indirectly, such as an enzyme whose products are detectable (e.g., luciferase, β-galactosidase, peroxidase, etc.). Antibodies of the invention may be attached to a solid support.

Nucleic acid amplification methods

Nucleic acid in a sample can conveniently and sensitively be detected by nucleic acid amplification techniques such as PCR, SDA, SSSR, LCR, TMA, NASBA, T7 amplification, etc. The technique preferably gives exponential amplification. A preferred technique for use with RNA is RT-PCR (e.g., see chapter 15 of ref 83). The technique may be quantitative and/or real-time.

Amplification techniques generally involve the use of two primers. Where an influenza virus target sequence is single-stranded, the techniques generally involve a preliminary step in which a complementary strand is made in order to give a double-stranded target, thereby facilitating exponential amplification. The two primers hybridize to different strands of the double-stranded target and are then extended. The extended products can serve as targets for further rounds of hybridization/extension. The net effect is to amplify a template sequence within the target, the 5′ and 3′ termini of the template being defined by the locations of the two primers in the target.

The invention provides a kit comprising primers for amplifying a template sequence contained within an influenza virus nucleic acid target, the kit comprising a first primer and a second primer, wherein the first primer comprises a sequence substantially complementary to a portion of said template sequence and the second primer comprises a sequence substantially complementary to a portion of the complement of said template sequence, wherein the sequences within said primers which have substantial complementarity define the termini of the template sequence to be amplified.

The first primer and/or the second primer may include a detectable label (e.g. a fluorescent label, a radioactive label, etc.).

Primers may include a sequence that is not complementary to said template nucleic acid. Such sequences are preferably upstream of (i.e., 5′ to) the primer sequences, and may comprise a restriction site [84], a promoter sequence [85], etc.

Kits of the invention may further comprise a probe which is substantially complementary to the template sequence and/or to its complement and which can hybridize thereto. This probe can be used in a hybridization technique to detect amplified template.

Kits of the invention may further comprise primers and/or probes for generating and detecting an internal standard, in order to aid quantitative measurements [86].

Kits of the invention may comprise more than one pair of primers (e.g., for nested amplification), and one primer may be common to more than one primer pair. The kit may also comprise more than one probe.

The template sequence is preferably at least 50 nucleotides long (e.g., 60, 70, 80, 90, 100, 125, 150, 175, 200, 250, 300, 400, 500, 600, 700, 800, 900, 1000, 1250, 1500, 2000, 3000 nucleotides or longer). The length of the template is inherently limited by the length of the target within which it is located, but the template sequence is preferably shorter than 500 nucleotides (e.g., 450, 400, 350, 300, 250, 200, 175, 150, 125, 100, 90, 80, 70, or shorter).

The template sequence may be any part of an influenza virus genome sequence.

The invention provides a process for preparing a fragment of a target sequence, wherein the fragment is prepared by extension of a nucleic acid primer. The target sequence and/or the primer are nucleic acids of the invention. The primer extension reaction may involve nucleic acid amplification (e.g., PCR, SDA, SSSR, LCR, TMA, NASBA, etc.).

### Pharmaceutical Compositions

The invention provides a pharmaceutical composition comprising an antiviral, nucleic acid, polypeptide and/or antibody of the invention. Compositions of the invention optionally further comprise an immunomodulatory compound effective to inhibit natural killer cell activity. The invention also provides their use as medicaments (e.g., for prevention and/or treatment of type 1 diabetes and/or pancreatitis), and use of the components in the manufacture of medicaments for treating type 1 diabetes and/or pancreatitis. The invention also provides a method for raising an immune response, comprising administering an immunogenic dose of nucleic acid and/or polypeptide of the invention to an animal (e.g., to a patient).

Pharmaceutical compositions encompassed by the present invention include as active agent, an antiviral, nucleic acid, polypeptide, antibody, and/or immunomodulatory compound effective to inhibit natural killer cell activity of the invention disclosed herein, in a therapeutically effective amount. An “effective amount” is an amount sufficient to effect beneficial or desired results, including clinical results. An effective amount can be administered in one or more administrations. For purposes of this invention, an effective amount is an amount that is sufficient to palliate, ameliorate, stabilize, reverse, slow or delay the symptoms and/or progression of type 1 diabetes and/or pancreatitis.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers (e.g., insulin production). Therapeutic effects also include reduction in physical symptoms. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg of the compositions of the present invention in the individual to which it is administered.

A pharmaceutical composition can also contain a pharmaceutically acceptable earner. A thorough discussion of such carriers is available in reference 87.

Once formulated, the compositions contemplated by the invention can be (1) administered directly to the subject (e.g., as nucleic acid, polypeptides, small molecule antivirals, and the like); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

### General

The term “comprising” encompasses “including” as well as “consisting,” e.g., a composition “comprising” X may consist exclusively of X or may include something additional, e.g., X+Y.

The term “about” in relation to a numerical value x is optional and means, for example, x±10%.

The word “substantially” does not exclude “completely,” e.g., a composition which is “substantially free” from Y may be completely free from Y. Where necessary, the word “substantially” may thus be omitted from the definition of the invention.

### BRIEF DESCRIPTION OF DRAWINGS

FIGS. 1A, 1B, and 1C show glucose and lipase plasmatic concentrations for groups A (receiving H7N1 A/turkey/Italy/3675/1999, FIG. 1A), B (receiving H7N3A/turkey/Italy/2962/2003, FIG. 1B) and K (control, FIG. 1C). ID: identification number; n.d.: not done; eut: euthanized in order to collect the samples for histology and immunohistochemistry at designated days post-infection or due to the end of the experiment; columns highlighted in dark grey: days in which only subjects with high lipase concentration were tested with Glucocard® strips (upper limit 34 mmol/L); columns highlighted in light grey: particularly relevant data.

FIGS. 2A and 2B show Kaplan-Meier analyses for the appearance of hyperlipasemia (FIG. 2A) and hyperglycaemia (FIG. 2B) (plasma glucose >27.78 mmol/L,) between the mock, H7N1 and H7N3 infected turkeys. Differences were tested using the log rank statistic. Bar graphs: frequency of events in relation to hyperlipasemia, hyperglycaemia and viraemia.

FIG. 3 shows a turkey pancreas section (normal tissue). Acinar cells containing zymogen granules in their cytoplasm are evident, associated with two nests of normal islet cells and a ductal structure.

FIG. 4 shows a turkey pancreas section 7 days post infection. Diffuse and severe necrosis of acinar cells (arrows) with severe inflammatory infiltrate (*).

FIG. 5 shows a turkey pancreas section. Most of the pancreas is replaced by foci of lymphoid nodules and fibrous connective tissue and lymphoid nodules with some ductular proliferation.

FIG. 6 shows a turkey pancreas section 4 days post infection. Immunohistochemistry for avian influenza nucleoprotein (NP). Positive nuclei and cytoplasm are evident in necrotic acinar cells and in the ductal epithelium.

FIGS. 7A, 7B, 7C, and 7D show replication kinetics in pancreatic cell lines of A/New Caledonia/20/99 (H1N1) and A/Wisconsin/67/2005 (H3N2) in hCM and HPDE6 cells. hCM and HPDE6 cells were infected with each virus at an MOI=0.001. At 24, 48 and 72 hours post-infection, supernatants from three infected and one mock-infected control well were harvested for virus isolation and qRRT-PCR analysis. FIG. 7A shows virus Isolation results of H1N1 in hCM and HPDE6. FIG. 7B shows qRRT-PCR results of H1N1 in hCM and HPDE6. FIG. 7C shows virus Isolation results of H3N2 in hCM and HPDE6. FIG. 7D shows qRRT-PCR results of H3N2 in hCM HPDE6. All results represent means plus standard deviations of three independent experiments.

FIGS. 8A, 8B, 8C, 8D, 8E, 8F, 8G, and 8H show Western blot analyses of H1N1 (FIGS. 8A and 8B) and H3N2 (FIGS. 8E and 8F) influenza virus NP expression (56KDa) in hCM and HPDE6 cells. Samples were collected before infection (t0) and 24 (t24), 48 (t48) and 72 (t72) hours post-infection. Beta-actin (42 KDa) was used as loading control in order to assure that the same amount of proteins was tested for each sample (FIGS. 8C, 8D, 8G, and 8H).

FIGS. 9A, 9B, 9C, and 9D show nuclear staining of HPDE6 negative control (20X) (FIG. 9A). Cells were DAPI stained to reveal bound to DNA and with Evans Blue as contrast. FIG. 9B shows HPDE6 at 24 h post-infection (20X). Influenza virus NP protein derived from viral infection was observed (center of image). FIG. 9C shows HCM negative control. FIG. 9D shows hCM at 24 hours post-infection (20X), Influenza virus NP protein derived from viral infection was observed as brightly coloured cells in the center of the image.

FIGS. 10A, 10B, 10C, and 10D shows RRT-PCR data for M gene in human pancreatic islets: Two-way quadratic prediction plot with CIs (confidence interval) for RRT-Real time Ct values obtained from H1N1 (FIGS. 10A and 10C) and H3N2 in pancreatic islets (FIGS. 10B and 10D) 4.8×10³ PFU/well pancreatic islet cell infection. For each virus are represented the Ct trend in pancreatic islet pellets and supernatants from the day of infection (to) until day 10 (t₅) in presence (first column) or absence (second column) of TPCK and as an average of the previous two conditions (third column).

FIG. 11 shows Western Blot NP results for H1N1 infection with (TPCK+) or without (TPCK−) trypsin in pancreatic islets. Influenza virus nucleoprotein was visualized as a band of 56 KDa.

FIGS. 12A, 12B, and 12C. Viral RNA detection by in situ hybridization in human pancreatic islet. Islets were infected with H1N1 and H3N2 adding 100 μl of viral suspension containing viral dilution of 4.8×10³ pfu/well. Mock uninfected islets were left as a negative control. FIG. 12A: Two days after infection the presence of the virus RNA molecules was detected on cyto-embedded pancreatic islets upon addition of the Fast Red alkaline phosphatase substrate due to the formation of a coloured precipitate. Bound viral mRNA was then visualized using either a standard bright field or a fluorescent microscope (40X). Arrows: viral mRNA positive cells. FIGS. 12B and 12C: Five days after infection multiplex fluorescence-based in situ hybridization was performed and after disaggregation, islet cells were cytocentrifuged onto glass slides. Virus RNA, insulin, amylase and CK19 positive cells were assessed with a Carl Zeiss Axiovcrt 135TV fluorescence microscope. Quantification was performed using the IN Cell Investigator software. Each dot represents the percentage of positive cells quantified on one systematically random field. Results from two experiments performed are shown. Mann-Whitney U test was used for statistical analysis.

FIG. 13. Virus RNA and insulinlamylase/CK19 localisation. Figure shows multiplex histology data. Islets were infected with H1N1 and H3N2 adding 100 μl of viral suspension containing viral dilution of 4.8×10³ pfu/well. Five days after infection multiplex fluorescence-based in situ hybridization was performed as described above. Left panels: the red signal corresponds to the presence of influenza virus RNA, the green signal corresponds to the presence of insulin, amylase or CK18 transcripts (63x). White arrow: double positive cells. Right panel: Virus RNA, insulin, amylase and CK19 positive cells were assessed with a Carl Zeiss Axiovert 1TV fluorescence microscope. Quantification was performed using the IN Cell Investigator software. Each dot represents the percentage of positive cells quantified on one systematically random field. Results from two experiments performed are shown.

FIGS. 14A and 14B. Islet survival and insulin secretion after infection with Human Influenza A Viruses. Islets were infected with H1N1 and H3N2 adding 100 μl of viral suspension containing viral dilution of 4.8×10³ pfu/well. Mock uninfected islets were left as a negative control. The viabilities of pancreatic islets was evaluated 2, 5 and 7 days after infection. FIG. 14A shows light microscopy appearance of paraffin embedded islets 5 days after infection (20x) (upper). The viability (lower) was assessed using Live/Dead assay. Quantification was performed using the IN Cell Investigator software. Each dot represents the percentage of dead cells quantified on one random field. Results from two experiments (10 field each) are shown. FIG. 14B shows insulin secretion of isolated islets after culture for two days in the presence or in the absence of Human Influenza A Viruses. The figure shows insulin release after stimulation with glucose (2 to 20 mM) data are expressed as insulin secretion index calculated as the ratio between insulin concentration at the end of high glucose incubation and insulin concentration at the end of low glucose incubation, n=2.

FIGS. 15A and 15B. Cytokine/chemokine expression profile modification induced by Human Influenza A Viruses infection. Islets were infected with H1N1 and H3N2 adding 100 μl of viral suspension containing two viral dilutions of 4.8×10³ or 4.8×10² pfu/well. Mock uninfected islets were left as a negative control. Samples were collected every 48 hours from day of infection (t₀) until day 10 (t₁₀). The supernatant was collected and assayed for 50 cytokines.

FIG. 15A shows virus induced modification in islet cytokine/chemokine profile. Data are expressed as maximum fold increase for each factor detected during the culture respect mock infected islet (n=2). Dotted line: fivefold increase threshold. FIG. 15B shows IFN-gamma-inducible chemokines CXCL9/MIG, CXCL10/IP-10 concentration during ten day culture in the presence or in the absence of H1N1 and H3N2.

FIG. 16. Influenza virus M gene detection by RRT-PCR in pancreas and lungs of infected birds.

FIGS. 17A, 17B, 17C, 17D, and 17E. Immunohistochemistry for insulin. Pancreas, turkey. Representative islet structures before and after H3N7 at different time points.

FIG. 18. Receptor distribution profiles. Expression of alpha-2,3 and alpha-2,6-linked Sialic acid receptors on hCM, HPDE6 and MDCK cells. Shaded areas represent cells labelled with alpha-2,3 or alpha-2,6-specific lectins while unfilled areas represent unlabelled control cells. A minimum of 5,000 events were recorded per cell line.

FIGS. 19A, 19B, 19C, and 19D. Avian influenza virus replication kinetics in pancreatic cell lines. Replication kinetics of A/turkey/Italy/3675/1999 (H7N1) and A/turkey/Italy/2962/2003 (H7N3) in hCM and HPDE6 cells. hCM and HPDE6 cells were infected with each avian virus at an MOI=0.01 and at 24, 48 and 72 hours post-infection supernatants from three infected and one mock-infected control well were harvested for virus isolation and qRRT-PCR. (FIG. 19A) qRRT-PCR results of H7N1 in hCM and HPDE6. (FIG. 19B) qRRT-PCR results of H7N3 in hCM and HPDE6. (FIG. 19C) Virus isolation results of H7N1 in hCM and HPDE6. (FIG. 19D) Virus isolation results of H7N3 in hCM and HPDE6. All results represent means plus standard deviations of three independent experiments.

FIGS. 20A, 20B, 20C, and 20D. Immunofluorescence targeting the viral NP protein in pancreatic cell lines. (FIG. 20A) hCM negative control. (FIG. 20B) hCM at 24 hours post-infection (20X). (FIG. 20C) Nuclear staining of HPDE6 negative control (20X). The blue color corresponds to DAPT dye bound to DNA, while the red one is due to the Evans Blue contrast. (FIG. 20D) HPDE6 at 24 h post-infection (20X). The green signal corresponds to the presence of influenza virus NP protein derived from viral infection.

FIG. 21. Selected cytokines/chemokines, limits of detection and the coefficients of variability (intra Assay % CV and inter Assay % CV)

FIGS. 22 Group A and Group B. Viral shedding and viremia data.

### MODES FOR CARRYING OUT THE INVENTION

Certain aspects of the present invention are described m greater detail in the non-limiting examples that follow. The examples are put forth so as to provide those of ordinary skill in the art with a disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g., amounts, temperature, etc.) but some experimental errors and deviations should be accounted for.

In this study the inventors explored the implications of influenza infection on pancreatic endocrine function in an animal model, and performed in vitro experiments aiming to establish the occurrence, extent and implications of influenza A virus infection in human cells of pancreatic origin. For the in vivo studies the inventors selected the turkey as a model because turkeys are highly susceptible to influenza infection and pancreatic damage is often observed as a post-mortem lesion. For the in vitro studies, the inventors selected A/New Caledonia/20/99 (H1N1) and A/Wisconsin/67/05 (H3N2), as these viruses have circulated for extensive periods in humans, and existing epidemiological data would be suitable for a retrospective study. These strains were used to infect both established human pancreatic cell lines (including human insulinoma and pancreatic duct cell lines) and primary culture of human pancreatic islets.

In Vivo Experiments

Influenza A viruses originate from the wild bird reservoir and infect a variety of hosts including wild and domestic birds. These viruses are also able to infect a relevant number of mammals, in which they may become established. Among the latter there are swine, equids, mustelids, sea mammals, canids, felids and humans. IAV cause systemic or non-systemic infection depending on the strain involved. The systemic disease occurs mostly in avian species and is known as Highly Pathogenic Avian Influenza (HPAI). It is characterized by extensive viral replication in vital organs and death within a few days from the onset of clinical signs in the majority of infected animals. The non-systemic form, which is by far the most common, occurs in birds and in mammals and is characterised by mild respiratory and enteric signs. To differentiate it from HPAI, in birds it is known as low pathogenicity avian influenza (LPAI). This different clinical presentation resides in the fact that non-systemic influenza A viruses are able to replicate only in the presence of trypsin or trypsin-like enzymes and thus their replication is believed to be restricted to the respiratory and enteric tract.

IAV of avian origin have a tropism for the pancreas [5,88,89,90]. Necrotizing pancreatitis is a common finding in wild and domestic birds infected with HPAI [91,92,93,94] and the systemic nature of HP AI is in keeping with these findings. In contrast, it is difficult to explain pancreatic colonisation by LP AI viruses, which is a common finding in chickens and turkeys experiencing infection [95,96,97].

The aim of this study was to establish whether two natural non-systemic avian influenza viruses obtained from field outbreaks, without prior adaptation, could cause endocrine or exocrine pancreatic damage following experimental infection of young turkeys.

### Animals

Sixty-eight female meat turkeys obtained at one day of age from a commercial farm were used in this study. Birds were housed in negative pressure, high efficiency particulate air (HEPA) filtered isolation cabinets for the duration of the experimental trial. Before carrying out the infection, animals were housed for 3 weeks to allow adaptation and growth, received feed and water ad libitum and were identified by means of wing tags.

Viruses

Two low pathogenicity avian influenza viruses (LPAI) isolated during epidemics in Italy were used for the experimental infection: A/turkey/Italy/3675/1999 (H7N1) and A/turkey/Italy/2962/2003 (H7N3). Both viruses had shown to cause pancreatic lesions in naturally infected birds. Stocks of avian influenza viruses were produced inoculating via the allantoic cavity 9-day-old embryonated specific pathogen free (SPF) chicken eggs. The allantoic fluid was harvested 48 hours post inoculation, aliquoted and stored at −80° C. until use. For viral titration, 100 μl of 10-fold diluted viral suspension were inoculated in SPF embryonated chicken eggs and the median embryo infectious dose (EID₅₀) was calculated according to the Reed and Muench formula.

### Experimental Design

Animals were divided into three experimental groups [A (H7N1), B(H7N3) and K (control)]. Groups A and B, each constituted 24 animals, which were infected via the oro-nasal route with 0.1 ml of allantoic fluid containing 10^{6.83} EID₅₀ of the A/turkey/Italy/3675/1999 (H7N1) virus and 10^{6.48} EID₅₀ of the A/turkey/Italy/2962/2003 (H7N3) virus respectively. Group K, constituted animals, which received via the oro-nasal route 0.1 ml of negative allantoic fluid as negative control. All birds were observed twice daily for clinical signs. On days 0, 3, 6, 9, 13, 15, 20, 23, 27, 31, 34, 41 and 45 p.i. blood was collected from the brachial vein of all animals using heparinized syringes in order to determine glucose and lipase levels in plasma. On days 2 and 3 post infection (p.i.), tracheal swabs were collected to evaluate viral replication. On day 3 p.i., blood was also collected to determine the presence of viral RNA in the blood. On days 4 and 7 p.i., two birds from each infected group were humanely sacrificed and the pancreas and the lung were processed for the detection of viral RNA and for histopathology and immunohistochemistry. Similarly, on days 8 and 17 p.i., one subject from each experimental group was euthanized and the pancreas was collected for histological and immunohistochemical studies. For this purpose the inventors selected hyperglycaemic subjects that had also shown an increase in lipase levels.

### Biochemical Analyses

Blood samples were collected in Gas Lyte® 23 G pediatric syringes containing lyophilized lithium heparin as anticoagulant. At each sampling, 0.3 ml of blood was collected and refrigerated at 4° C. until processed. To obtain plasma, samples were immediately centrifuged at 1500×g for 15 minutes at 4° C. To determine the levels of glucose and lipase in plasma, commercially available kits (Glucose HK and LIPC, Roche Diagnostics GmbH, Mannheim, Germany) were applied to the computerised system Cobas c501 (F. Hoffmann-La Roche Std, Basel, Switzerland). The Glucose HK test is based on an hexokinase enzymatic reaction. The linearity of the reaction is 0.11-41.6 mmoVL (2-750 mg/dL) and its analytic sensitivity is 0.11 mmol/L (2 mg/dL). The LIPC test is based on a colorimetric enzymatic reaction with a linearity of 3 a 300 U/L and an analytic sensitivity of 3 U/L.

### Molecular Tests

Tracheal swabs, blood samples and organs (pancreas and lungs) were tested for viral RNA by means of RRT-PCR for the identification of the influenza virus Matrix (M) gene.

RNA extraction

Viral RNA was extracted from 100 μl of blood using the commercial kit “NucleoSpin RNA II” (Macherey-Nagel) and from 50 μl of phosphate buffered saline (PBS) containing tracheal swabs suspension using the Ambion MagMax-96 Al-ND Viral RNA Isolation Kit for the automatic extractor. 150 mg of homogenized lung and pancreas tissues were centrifuged and viral RNA was extracted from 100 μl of clarified suspension using the NucleoSpin RNA II (Macherey-Nagel).

### One Step RRT-PCR

The isolated RNA was amplified using the published primers and probes from reference 98, targeting the conserved Matrix (M) gene of type A influenza virus. 5 μL of RNA were added to the reaction mixture composed by 300 nM of the forward and reverse primers (M25F and M124-R respectively), and 100 nM of the fluorescent label probe (M+64). The amplification reaction was performed in a final volume of 25 μL using the commercial kit QuantiTect Multiplex RT-PCR kit (Qiagen, Hilden, Germany). The PCR reaction was performed using the following protocol: 20 minutes at 50° C. and 15 minutes at 95° C. followed by 40 cycles at 94° C. for 45 sec and 60 ° C. for 45 sec. Target RNA transcribed in vitro were obtained using the Mega Short Script 7 (high yield transcription kit, Ambion), according to the manifacturer' s instructions, quantified by NanoDrop 2000 (Thermo Scientific) and used to create a standard calibration curve for viral RNA quantification. To check the integrity of the isolated RNA, the β-actin gene was also amplified using a set of primers in-house designed (primers sequences available upon request). The reaction mixture was composed by 300 nM of forward and reverse primer and IX of EvaGreen (Explera, Jesi, Italy). The amplification reaction was performed in a final volume of 254, using the commercial kit Superscript III (Invitrogen, Carlsbad, Calif.). The PCR reaction was performed using the following protocol: 30 minutes at 55° C. and 2 minutes at 94° C. followed by 45 cycles at 94° C. for 30 sec and 60° C. for 1 min.

### Histology and Immunohistochemistry

Formalin-fixed, paraffin-embedded pancreas sections were cut (3 μm thickness). Slides were stained with H&E (Histoserv, Inc., Germantown, Md.). Representative photos were taken with the SPOT ADVANCED software (Version 4.0.X, Diagnostic Instruments, Inc., Sterling Heights, Mich.). The reagents and methodology for Influenza THC were: Polyclonal Antibody Anti- type A Influenza Virus Nucleoprotein, Mouse-anti-Influenza A (NP subtype A, Clone EVS 238, European Veterinary Laboratory, 1:100 in PBS/2.5% BsA, for 1 hour at RT ; secondary antibody Goat-anti-mouse IgG2a HRP (Southern Biotech) 1/200 in PBS/2.5% BSA, for 1 hour at RT; Antigen retrieval was performed incubating the slides for 10′ at 37° C. in trypsin (Kit Digest-all; Invitrogen); Endogenous peroxidase were blocked with 3% H₂O₂, for 10′ at RT, before incubation with primary antibody slides a blocking step was performed with PBS/5% BSA for 20′ at RT. DAB was applied as chromogen (Dakocytomation, ref. code K3468). IHC for insulin and glucagone: Polyclonal Guinea Pig Anti-Swine Insulin, 1:50 (A0564 Dako, Carpinteria, Calif.); Polyclonal Rabbit Anti-Glucagon, 1:200 (NCL-GLUC, Novocastra, Newcastle, UK) using as a detection system, the En Vision Ap (DAKO K1396, Carpinteria, Calif.) and nuclear fast Red (DAKO K1396) for the Influenza A staining; En Vision+System-HRP Labelled polymer Anti-Rabbit (K4002, Dako, Carpinteria, Calif.) and DAB (K3468, Dako, Carpinteria, Calif.) for Insulin and Glucagon staining.

### In Vitro Assays

The aims of these experiments were to establish whether human influenza viruses can grow on human primary and established cell lines derived from the human pancreas, and the effect of their replication on primary cells.

### Cell Lines

Maclin Darby Canine Kidney (MDCK) cells were maintained in Alpha's Modified Eagle Medium (AMEM, Sigma) supplemented with 10% Foetal Bovine Serum (FBS), 1% 200 mM L-glutamine and a 1% penicillin/streptomycin/nystatin (pen-strep-nys) solution. The human insulinoma cell line CM [99] and immortalized human ductal epithelial cell line HPDE6 [100] were maintained in RPMI (Gibco) supplemented with 1% L-glutamine, 1% antibiotics and FBS (5% and 10%, respectively). MDCKs and HPDE6 were passaged twice weekly at a subcultivation ratio of 1:10 and 1:4, while CM were split three times per week at a ratio of 1 :4. All cells were maintained in a humidified incubator at 37C with 5% CO₂.

### Primary Cells

Pancreatic islets were isolated and purified at San Raffaele Scientific Institute from pancreases of multiorgan donors according to Ricordi's method. Islet preparations with purity >80%±8% (mean±SD, n=6) not suitable for transplantation, were used after approval by the local ethical committee. Cells were seeded in 24 well plates and 25 cm2 flasks at 150 islets/ml and maintained in final wash culture medium (Mediatech, Inc., Manassas, Va.) medium at 37° C. with 5% CO₂.

### Sialic Acid Receptor Characterization on CM and HPDE6 Cells

The presence of alpha-2,3 and alpha-2,6-linked sialic acid residues was determined via flow cytometry. Following trypsinization, 1×10⁶ cells washed twice with PBS-10 mM HEPES (PBS-HEPES), for 5 minutes at 1200 RPM, and then treated with an Avidin/Biotin blocking kit (Vector Laboratories, USA) as per manufacturer's instructions, with cells incubated for 15 minutes with 100 μl of each solution. Alpha-2,3 and alpha-2,6 sialic acid linkages, respectively, were detected by incubating cells for 30 minutes with 100 μl of biotinylated Maackia amurensis lectin II (Vector Laboratories) (5 μg/ml) followed by 100 μl of PE-Streptavidin (BD Biosciences) (10 μg/ml) for 30 minutes at 4C in the dark, or with 100 μl of Fluorescein conjugated Sambucus nigra lectin (Vector Laboratories) (5 μg/ml). Cells were washed twice with PBS-HEPES between all blocking and staining steps and resuspended in PBS with 1% fonnalin prior to analysis. To confirm specificity of lectins, cells were pre-treated with 1 U per mL of neuraminidase from Clostridium peifringens (Sigma) for one hour prior to the avidin/biotin block. Samples were analyzed on a BD Facscalibur or the BD LSR II (BD Biosciences) and a minimum of 5,000 events were recorded.

### Viruses and Viral Titration

Stocks of A/New Caledonia/20/99 (H1N1) and A/Wisconsin/67/05 (H3N2), referred as H1N1 and H3N2 respectively, were produced in cell culture or in embryonated chicken eggs. Viruses were titrated by standard plaque assay.

To propagate IAV, monolayer cultured MDCK cells were washed twice with PBS and infected with A/NewCaledonia/20/99 (H1N1) or A/Wisconsin/67/05 (H3N2) at an MOI of 0.001. After virus adsorption for 1 h at 35° C., the cells were washed twice and incubated at 35° C. with DMEM without serum supplemented with TPCK-treated trypsin (1 μg/ml, Worthington Biomedial Corporation, Lakewood, N.J., USA). Supernatants were recovered forty-eight hours post-infection. Low Pathogenicity avian influenza viruses (LPAI) H7N1 A/turkey/Italy/3675/1999 and H7N3 A/turkey/Italy/2962/2003 isolated during epidemics in Italy were grown in 9-day-old embryonated specific pathogen free (SPF) chicken eggs as described in section 2.1.2. For viral titration, plaque assays were performed as previously described [101]. Briefly, MDCK monolayer cells, plated in 24-well plates at 2.5×10⁵ cells/well, were washed twice with DMEM without serum, and serial dilutions of virus were adsorbed onto cells for 1 hour. Cells were covered with MEM 2X—Avicel (FMC Biopolymer, Philadelphia, Pa., USA) mix supplemented with TPCK-treated trypsin (1 μg/ml). Crystal violet staining was performed 48 hours post-infection and visible plaques were counted.

### Virus Replication Kinetics in Pancreatic Cell Lines

Semi-confluent monolayers of HPDE6 and CM cells seeded on 24-well plates were washed twice with PBS and then infected at an MOI of 0.001 using 200 μl of inoculum per well. Inoculum was removed after one hour of absorption and replaced with 1 ml of serum-free media containing 0.05 μg/l TPCK-Trypsin (Sigma). At 1, 24, 48 and 72 hours post-infection supernatants from three infected wells and one control well were harvested, and viral titres were determined by virus isolation using the 50% tissue culture infectious dose (TCID₅₀) assay as well as by Real Time RT-PCR detection of the Matrix gene. All replication kinetics experiments were repeated three times.

### TCID₅₀.

Confluent monolayers of MDCK cells seeded onto 96-well plates were washed twice in serum-free medium and inoculated with 50 μl of 10-fold serially diluted samples in serum free MEM. After one hour of absorption an additional 50 μl of serum-free media containing 2 μg/ml TPCK-Trypsin was added to each well. CPE scores were determined after three days of incubation at 37° C. by visual examination of infected wells on a light microscope. The TCID₅₀ value was determined using the method of Reed and Muench.

### Growth Assay in Pancreatic Islets

Islets were infected with H1N1 and H3N2 influenza viruses adding 4.8×10² or 4.8×10³ pfu/well. Viral growth was performed with and without the addition of TPCK trypsin (SIGMA®) (1 μg/ml). Uninfected islets were left as a negative control. Samples were collected every 48 hours from day of infection (t₀) until day 10 (t₅). Each sample was centrifuged at 150 g for 5 minutes. The supernatant was collected and stored at −80° C. for quantitative Real Time PCR, virus titration and cytokine expression profile. The pellet was washed twice with PBS, stored at −80° C. and subsequently processed for Real Time PCR, Western Blot and virus titration in MDCK cells, see above). All pellets and supernatants were tested for Real Time PCR in triplicate.

### Detection of Viral RNA (Rom Pancreatic Tissue

The total RNAs from pancreatic islet pellets and supernatants were isolated using the commercial kit “NucleoSpin RNA II” (Macherey-Nagel) according to the manifacturer' s instructions. RNAs were eluted in 60 μl of elution buffer and tested using One step RRT-PCR for influenza Matrix gene (see below) to evaluate the viral growth.

A quadratic regression model (Ct=β₀+β₁TPCK-trypsin+β₂time+β₃time²+β₄time·TPCK-trypsin β₅time² TPCK-trypsin) for each viruses and specimen was used to analyse the trend of Ct value over time. The influence of TPCK presence and the interaction between its presence and time point was evaluated. The regression model took into account the influence of the intra-group correlation among repeated measurements for each observed time in the confidence intervals (CIs) calculation. A residuals post-estimation analysis was performed to verify the validity of the model.

### One Step RRT-PCR

Quantitative Real Time PCR, targeting the conserved Matrix (M) gene of type A influenza virus, was applied according to the protocol described in section 2.1.5 above. To check the integrity of the isolated RNA, the β-actin gene was also amplified using primers and probe previously described [102]. The reaction mixture was composed by 400 nM of forward and reverse primer (Primer-beta act intronic and Primer-beta act reverse respectively) and 200 nM of the fluorescent label probe (5′-Cy5 3′-BHQ1). The amplification reaction was performed in a final volume of 25 μL using the commercial kit QuantiTect Multiplex® RT-PCR kit (Qiagen, Hilden, Germany). The PCR reaction was using the following protocol: 20 minutes at 50° C. and 15 minutes at 95° C. followed by 45 cycles at 94° C. for 45° C. and 55° C. for 45 sec.

### Western Blot Analysis

Cellular pellets were resuspended in lysis buffer (50 mM Tris-HCl, pH 8; 1.0% SDS; 350 mM NaCl; 0.25% Triton-X; proteases inhibitor cocktail) then mixed and incubated on ice for 30 minutes. The suspension was sonicated three times for 5 minutes each and then centrifuged at maximum speed for 10 minutes. Bradford test was performed in order calculate the total protein concentration for each sample. Based on this calculation the same amount of protein/sample was treated in dissociation buffer (50 mM Tris-Cl, pH 6.8; 5% β-mercaptoethanol, 2% SDS, 0.1% bromophenol blue, 10% glycerol) for 5 minutes at 96° C. and then electrophoresed in 12% polyacrilamide gels using running buffer (25 mM Tris, 250 mM glycine, 0.1% SDS). Following SDS-PAGE the proteins were transferred from the gel onto immuno-blot PVD membranes (Bio-Rad) by electroblotting with transfer buffer (39 mM glycine, 48mM Tris base, 0.037% SDS, 20% methanol). Membranes were washed with PBS and then incubated overnight at 4° C. in 5% dried milk in PBS. After washing with PBS membranes were incubated for 1 h at room temperature under constant shaking in PBS containing 0.05% Tween-20 (SIGMA®), 5% blotting grade blocker non-fat dry milk (Bio-Rad) and mouse monoclonal Influenza A virus Nucleoprotein antibody (Abcam). Beta Actin antibody (Abcam) was used as loading control. After incubation with the primary antibody, membranes were exposed for 1 h to horseradish peroxidise-(HRP) rabbit polyclonal secondary antibody to mouse TgG (Abcam), followed by visualization of positive bands by ECL using Hyperfilm™ ECL (Amersham Biosciences).

### Visualisation of Viral Growth in Pancreatic Cell Lines

HPDE6 and hCM cells were grown in slides to 80% confluence and infected with either H1N1 or H3N2 viruses at an M.O.I. of 0.1 with 0.05 mg/ml of TPCK. Cells were fixed and permeabilized at 0, 24, 48 and 72 h p.i. with chilled acetone (80%). After blocking with PBS containing 1% BSA, the cells were incubated for 1 h at 37° C. in a humidified chamber with mouse monoclonal to influenza A virus nucleoprotein—FITC conjugated (Abcam) in PBS containing 1% BSA and 0.2% Evan's Blue. The staining solution was decanted and the cells were washed three times. Nuclei of negative control cells were stained with DAPI (SIGMA), then washed with PBS and observed under UV light.

### In Situ Visualisation of Viral RNA in Pancreatic Islets

To visualize viral RNA localized within cells, purified human pancreatic islets were harvested at 2, 5 and 7 days post infection. Islets were then incubated for 24 h in methanol-free 10% formalin, deposited at the bottom of flat-bottomed tubes, embedded in agar to immobilize them, dehydrated, and finally embedded in paraffin. Islet samples were sectioned at 4 mm. For co-ocalization experiments, islets were harvested 5 days post infection, enzymatically digested into single cells with a trypsin-like enzyme (12605-01, TrypLE™ Express, Invitrogen, Carlsband, California) and cytocentrifuged onto glass slides. In situ hybridization was performed using the Quantigene ViewRNA technique, based on multiple oligonucleotide probes and branched DNA signal amplification technology, according to the manufacturer instructions (Affymetrix, Santa Clara, Calif., USA). The probe set used was designed to hybridize the H1N1/A/New Caledonia/20/99 virus (GenBank sequence: DQ508858.1). Due to sequence homology in the region covered by the probes, the same set recognized also the H3N2 virus RNA as confirmed in pilot experiments. To identify cell types within islets the following Quantigene probes were used: insulin for beta cells (INS gene, NCBI Reference Sequence: NM_000207); alpha-amylase 1 for exocrine cells (AMY1A gene, NCBI Reference Sequence:NM_004038); cytokeratin 19 for duct cells (KRT19 gene, NCBI Reference Sequence: NM_002276). Quantification of cells positive for each probe was performed within 8 randomly chosen fields using the IN Cell Investigator software (GE Healthcare UK Ltd).

### Determination of Insulin Secretion in Infected Islets

Aliquots of 100 islet equivalents (uninfected or infected with H1N1/A/New Caledonia/20/99 and H3N2/A/Wisconsin/67/05) per column were loaded onto Sephadex G-10 columns with media at low glucose concentration (2mM) and preincubated at 37° C. for 1 hour. After preincubation, islet were exposed to sequential 1 hr incubations at low (2 mM) and high (20 mM) glucose concentration. Supernatants were collected with protease inhibitors cocktail (Roche Biochemicals, Indianapolis, Ind.) and stored at −80° C. at the end of each incubation. Insulin content was determined with an insulin enzyme-linked immunoassay kit (Mercodia AB, Uppsala, Sweden) following manufacter's instruction. Insulin secretion index were calculated as the ratio between insulin concentration at the end of high glucose incubation and insulin concentration at the end of low glucose incubation

### Cytokine Expression Profile

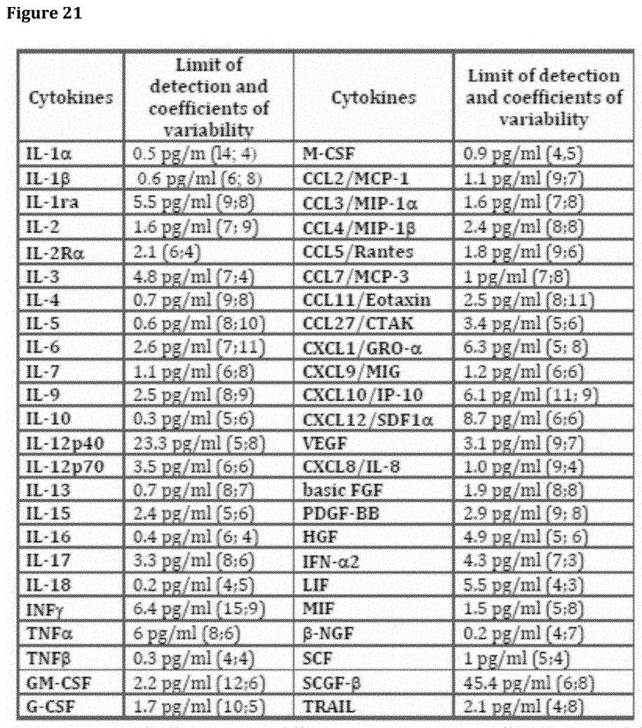

The capability of H1N1 and H3N2 viruses to induce cytokine expression in human pancreatic islets was measured using multiplex bead-based assays based on xMAP technology (Bio-Plex; Biorad Laboratories, Hercules, Calif., USA). The parallel wells of pancreatic were infected with viruses or were mock infected. The culture media supernatant was collected before and 2, 4, 6, 8, 10 days post infection and assayed for 48 cytokines. Selected cytokines, limits of detection and the coefficients of variability (intra Assay % CV and inter Assay % CV) of the cytokine/chemokine are shown in FIG. 21.

### Evaluation of Cell Death Following Infection (Live/Dead Assay)

The viability of islet cells after infection was measured using the live/dead cell assay kit (L-3224, Molecular Probes, Inc., Leiden, The Netherlands). The assay is based on the simultaneous determination of live and dead cells with two fluorescent probes. Live cells are stained green by calcein due to their esterase activity, and nuclei of dead cells are stained red by ethidium homodimer-1. Islets harvested after five days of culture were further enzymatically digested into single cells with trypsin-like enzyme (12605-01, TrypLE™ Express, Invitrogen, Carlsband, Calif.). According to manufacturer's instructions single cells were incubated with the labeling solution for 30 min at room temperature in the dark, cytocentrifuged onto glass slides, and assessed with a Carl Zeiss Axiovert 135TV fluorescence microscope. Analysis of dead cells were performed on cytospin preparations using the IN Cell Investigator software (GE Healthcare UK Ltd). Positive cells in each category were quantified with 10 systematically random fields.

### Statistical Analysis

Data were generally expressed as mean±standard deviation or median (Min-Max). Differences between parameters were evaluated using Student's T test when parameters were normally distributed, Mann Whitney U test when parameters were not normally distributed. Kaplan-Meier and/or Cox regression Analysis was used to analyze incidence of event during the time. A p value of less than 0.05 was considered an indicator of statistical significance. Analysis of data was done using the SPSS statistical package for Windows (SPSS Inc., Chicago, Ill., USA).

### RESULTS

### In Vivo Experiment

### Clinical Disease

Turkeys from both H7N1 [A] and H7N3 [B] challenged groups showed clinical signs typical of LPAI infection, such as conjunctivitis, sinusitis, diarrhoea, ruffled feathers and depression on day 2 p.i. Mild symptoms regressed by day 20 p.i. Only two subjects from group A showed sinusitis until day 30 p.i. Mortality rate was low in both groups: one subject of group A died on day 8 p.i. and one subject of group B died on day 19 p.i.

### Detection of Viral RNA

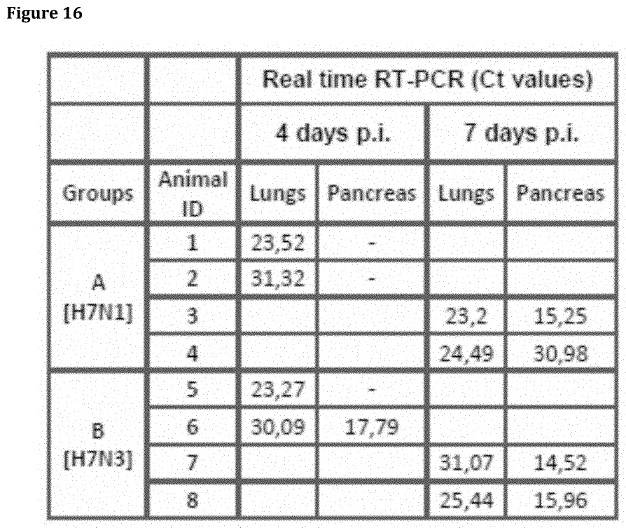

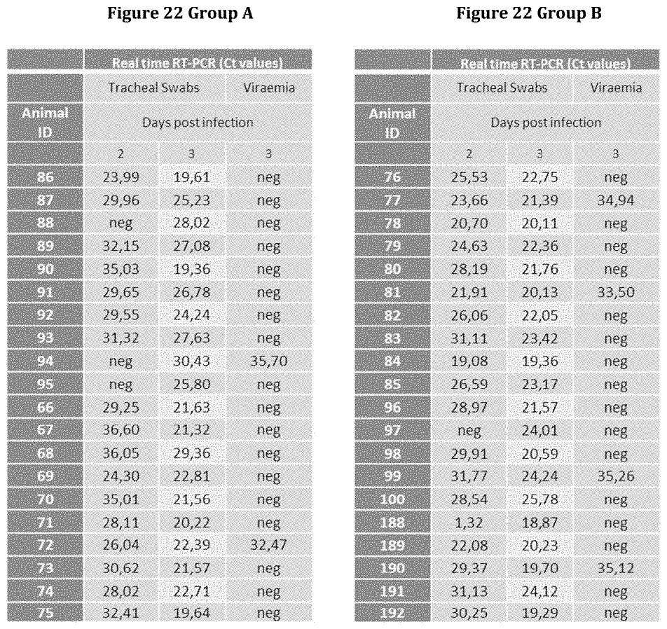

Viral RNA was detected from the tracheal swabs collected from 17/20 subjects infected with H7N1 and 19/20 subjects infected with H7N3 on day 2 and all animals on day 3 p.i. Viral RNA was also detected from the blood of two subjects of group A H7N1 and four subjects of group B H7N3 on day 3 p.i., (FIG. 22 Group A and Group B) and from the pancreas and lungs collected on days 4 and 7 p.i. (FIG. 16). No viral RNA was detected from the uninfected controls.

### Biochemical Analyses

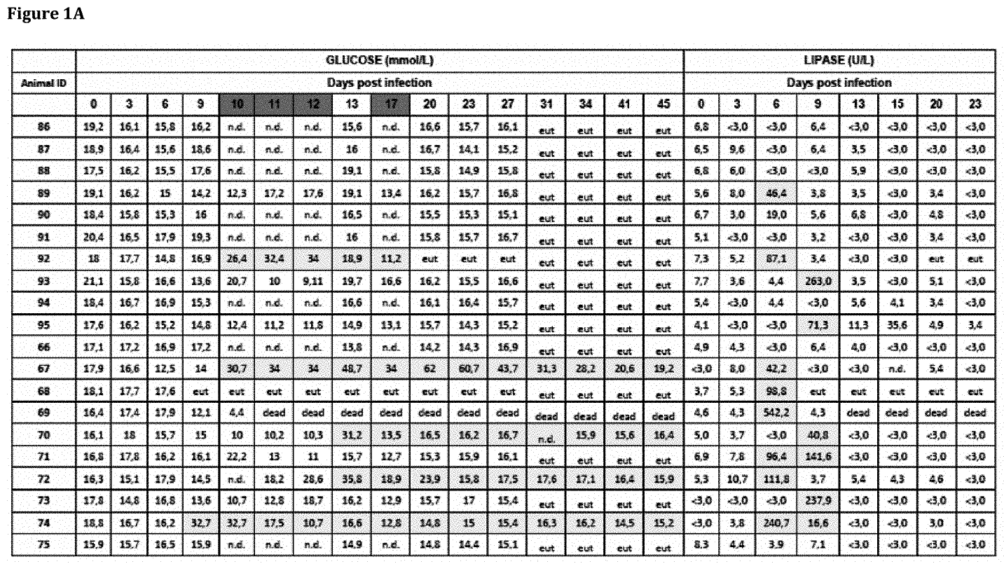

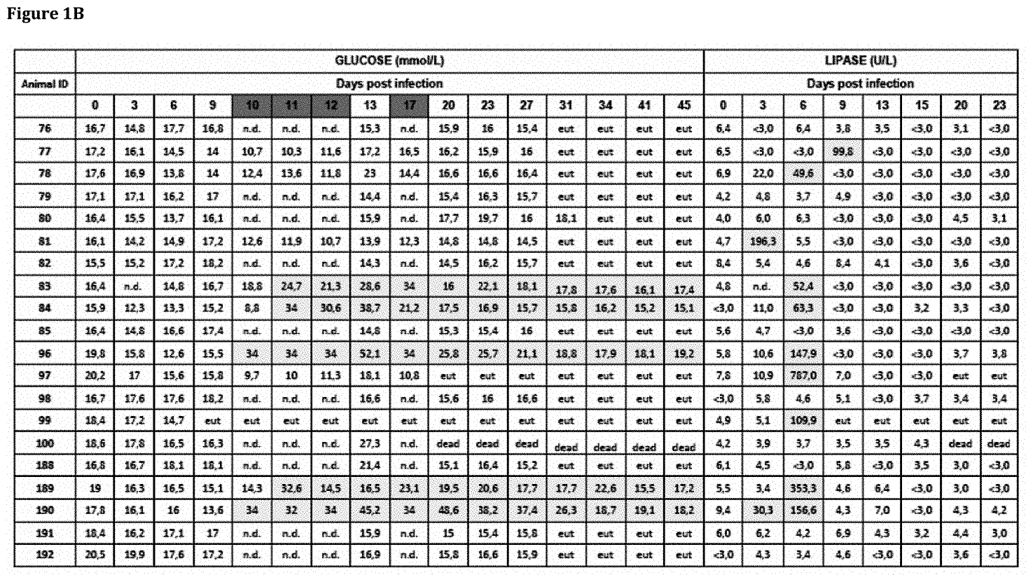

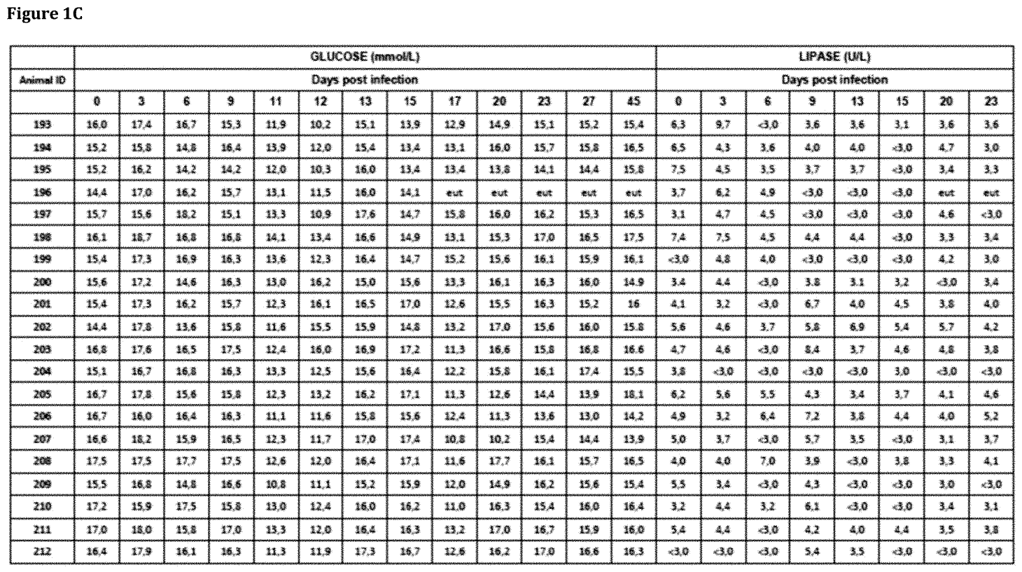

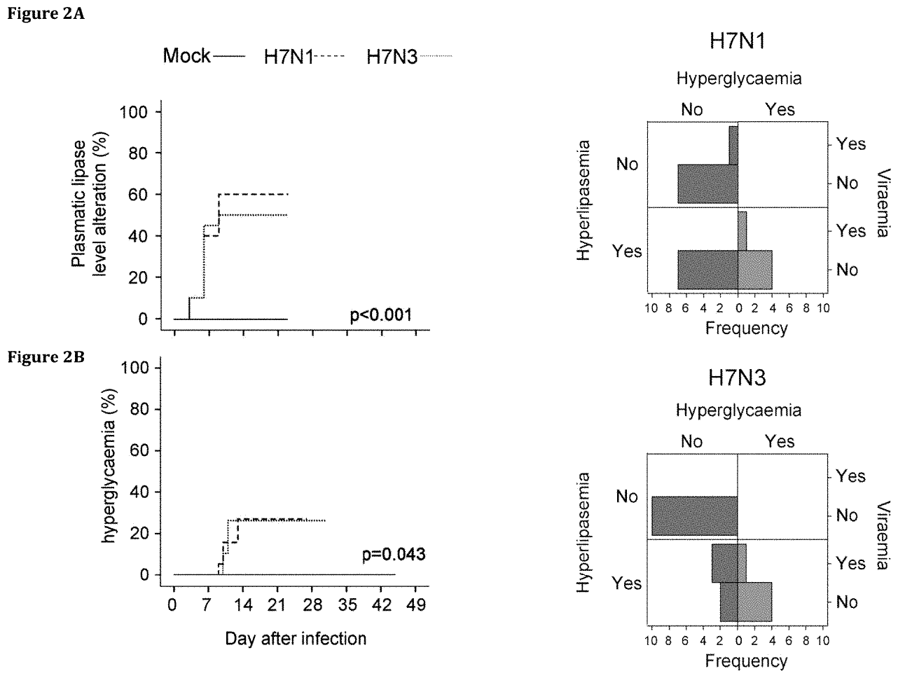

In blood samples collected intra-vitam to reveal metabolic alterations, a significant increase in plasmatic lipase levels (10 to 100 times the values of the control animals) was evident in H7N1 (12/20) and H7N3 (10/20) challenged turkeys between day 3 and 9 p.i. (FIGS. 2A and 2B) while none of uninfected controls showed modification of lipase levels (20/20; p<0.001, Pearson Chi-Square). A clear trend between the presence of viral RNA in blood at day 3 and the increase in lipase was evident in infected animals (Hazard Ratio 2.51 with 95% confidence interval 0.92 to 6.81; p 0.07). Lipase levels within the normal range were rapidly re-established in all cases, reason for which on day 23 p.i., it was decided to no longer evaluate this parameter on day 23 (FIGS. 1A, 1B, and 1C). After day 9 p.i. 5 animals of group A and 5 animals of group B developed hyperglycaemia (FIGS. 2A and 2B). Of these, two subjects maintained the hyperglycaemic status throughout the entire experiment while in all the other animals the levels of blood glucose returned similar to those of controls (FIGS. 1A, 1B, and 1C). A clear association between the increase in lipase between day 3 and 9 p.i. and the development of hyperglycaemia after day 9 p.i. was evident. In fact, hyperglycaemia was present only in the subjects who developed high lipase values post infection while never appeared in subject with normal lipase level (10/22 and 0/18 respectively, p=0.001) with a median time between hyperlipasemia and hyperglycaemia developments of 4.5 days (min-max: 3-7).

### Histopathology and Immunohistochemistry

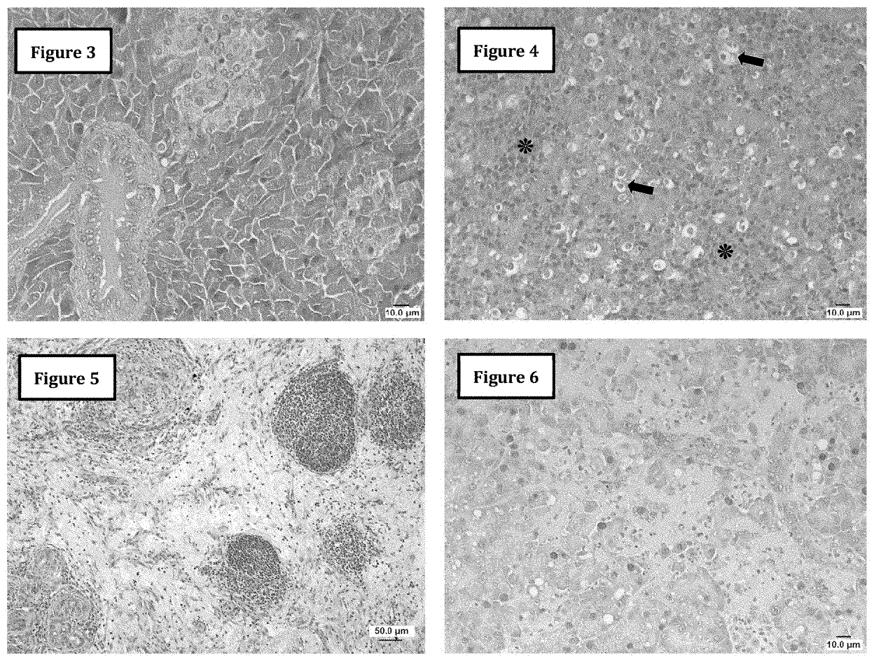

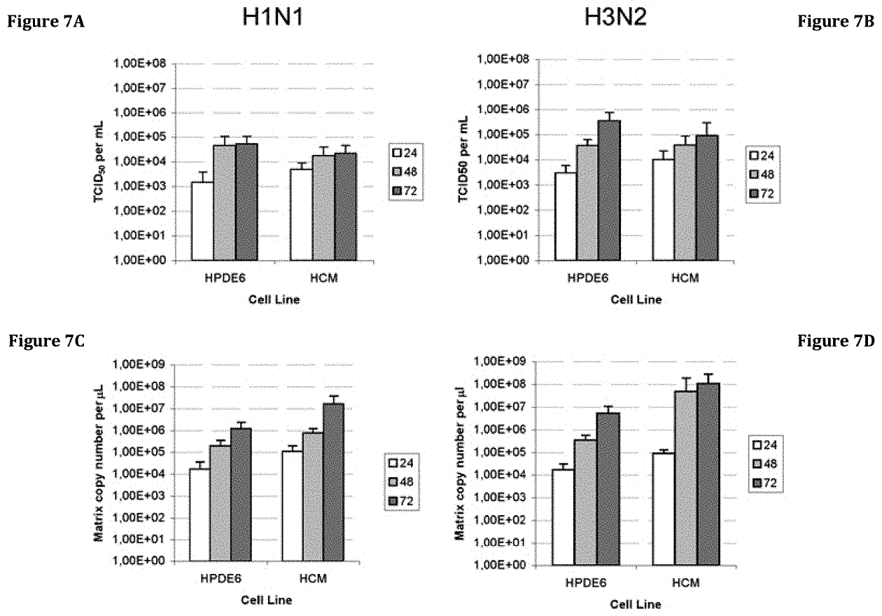

None of the control turkeys showed significant histological changes or positive immunohistological reactions against ATV (FIG. 3). In all infected birds, histopathologic lesions were evident, although markedly different in samples collected at different timings post infection. At early stages (day 4-8 p.i.), an acute pancreatitis with necrotic acinar cell, massive inflammatory infiltration composed of macrophages, heterophils, lymphocytes and plasmacells dominates over areas of healthy/uninvolved/spared tissue (FIG. 4). From day 8 p.i., these necrotic inflammatory lesions were gradually replaced by ductules and lymphocytic infiltration with mild degree of fibroplasia. At later stages (day 17 p.i) extensive fibrosis, with lymphoid nodules replaced pancreatic parenchyma and disruption of the normal architecture of the organ were evident (FIG. 5). Variable numbers of necrotic acinar cells were observed during all the experimental period. Obstructive ductal lesions were not seen in any case and stage.

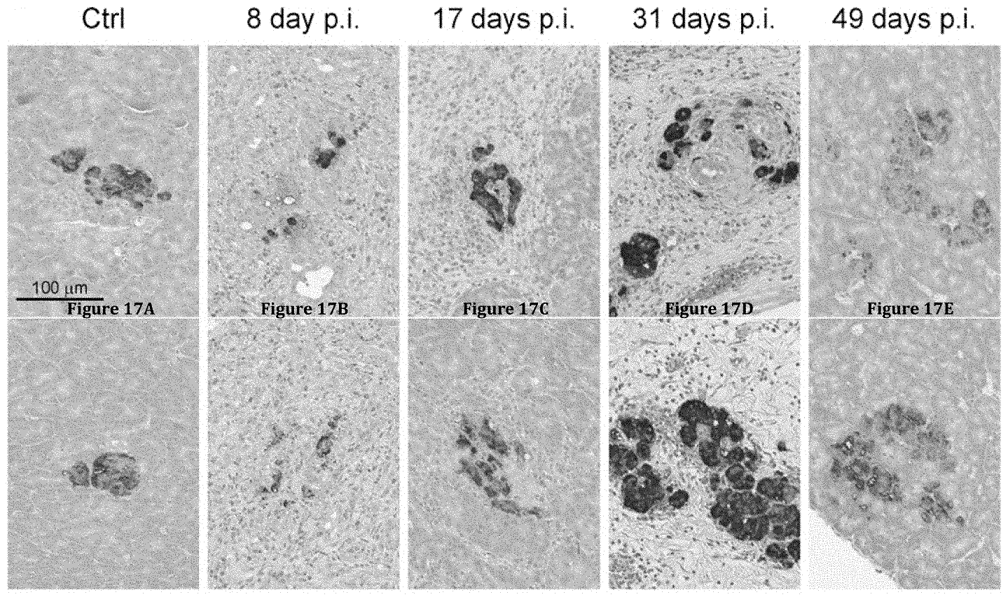

By immunohistological staining, degenerating and necrotic acinar cells showed specific reaction to virus nucleoprotein antigen antibody during the experimental period (FIG. 6). Some of the vascular endothelial cells also showed positive reaction, as well as occasional ductal epithelial cells. In uninfected controls the insulin positive tissues of the pancreas were scattered singly or in small groups of islets of various shapes and sizes in the intersititium of the exocrine part (FIG. 17A). At day 8 p.i. the normal structure of islets was partially destroyed and the number of islet cells was reduced. Remaining islets were smaller and distorted, with irregular outlines or dilated intra-islet capillaries; the number of cells staining for insulin was also reduced: these cells presented enlarged cytoplasm and sometimes appeared to have granular degeneration and even necrosis. Fibrous bands appeared inside the islet with islet fragmentation and dislocation of small and large clusters of endocrine cells (FIG. 17B). At day 17 p.i. separated large clusters of endocrine insulin positive cells were evident embedded in or close to the extensive fibrosis that replaced the acinar component (FIG. 17C). Beyond day 17 p.i. groups of very large (>200 μm in diameter), usually irregular, islet like areas of mainly insulin immunoreactivity were clearly present scattered in extensive acinar fibrosis (FIGS. 17D and 17E).

### In Vitro Experiment

Susceptibility of Human pancreatic cell lines to Human Influenza A Viruses

The susceptibility of endocrine (hCM, insulinoma) and ductal (HPDE6) cell lines to H1N1/A/New Caledonia/20/99 and H3N2/A/Wisconsin/67/05 infections were investigated.

### Receptor Distribution

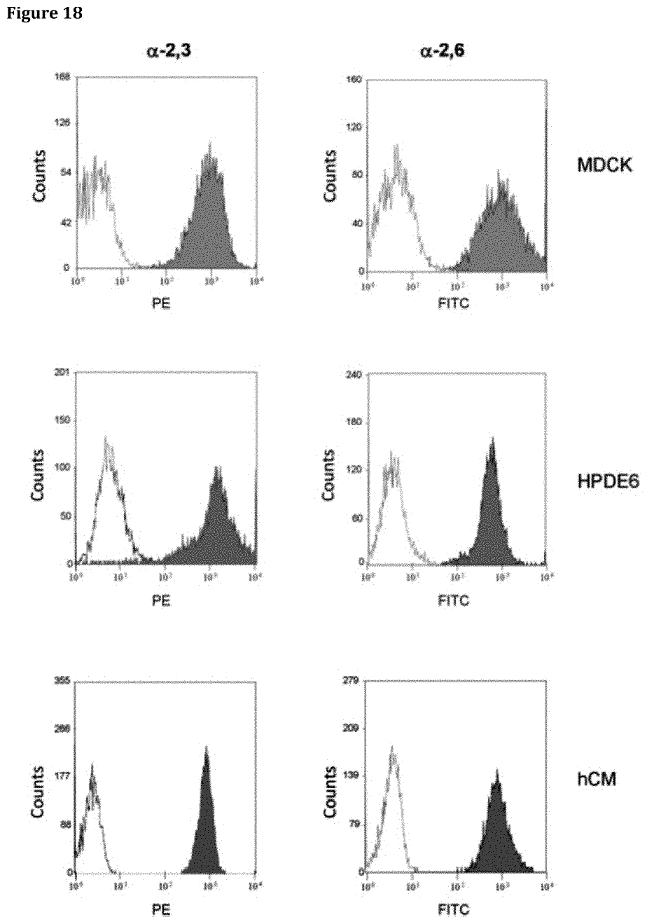

Lectin staining of both the hCM and HPDE6 cell lines revealed high levels of alpha-2,6 sialic acid-linked sialic acids molecules (required by human-tropic viruses) as well as alpha-2,3 linked residues (used by avian-tropic viruses). The mean peak intensities of hCM incubated with Maackia amurensis lectin II (alpha-2,3 specific) and Sambucus nigra lectin (alpha-2,6-specific), were nearly identical, at approximately 2.6×10⁴ for both receptors. HPDE6 also had high level expression of both receptor types, with 3.7×10⁴ for SNA and 1.6×10⁴ for MAA. MDCK cells were also included as a positive control line for both receptor types as these cells are widely used for the isolation of human and avian origin viruses. FACS analysis showed MDCKs expressed similar levels of alpha-2,3 receptors to the HPDE6, with mean peak intensity ncar 1.8×10⁴, while alpha-2,6 expression was equal to that of hCM, with a mean fluorescence at 2.5×10⁴. Therefore, both pancreatic cell lines can be said to express sialic acid receptors in levels comparable to MDCKs, and in the case of hCM expression of the human-virus receptors was even higher (FIG. 18). Pre-treatment of all cells with 1 U/ml of NA from Clostridium peifringens resulted in decreased fluorescence for both lectin types, confirming specificity (data not shown).

### Virus Replication Kinetics in Pancreatic Cell Lines

hCM and HPDE6 cells were infected with H1N1 and H3N2 viruses at a MOI=0.001. Visual examination of the infected cells by light microscopy revealed no cytopathic effect at any time point post-infection on hCM or HPDE6. TCID50 results revealed a continued increase in viral titres in HPDE6 over the 72 hour course, though the H1N1 viral titres were only slightly higher at 72 hours compared to 48 hours post-infection. In contrast, viral titres reached in hCM cells remained quite similar from 48 to 72 hours post-infection in the case of both H1N1 and H3N2 isolates (FIGS. 17A and 17C). An examination of viral RNA replication by qRRT-PCR showed a continued increase in viral replication up to 72 hours post-infection in both cell lines and for both viruses tested (FIGS. 17B and 17D).

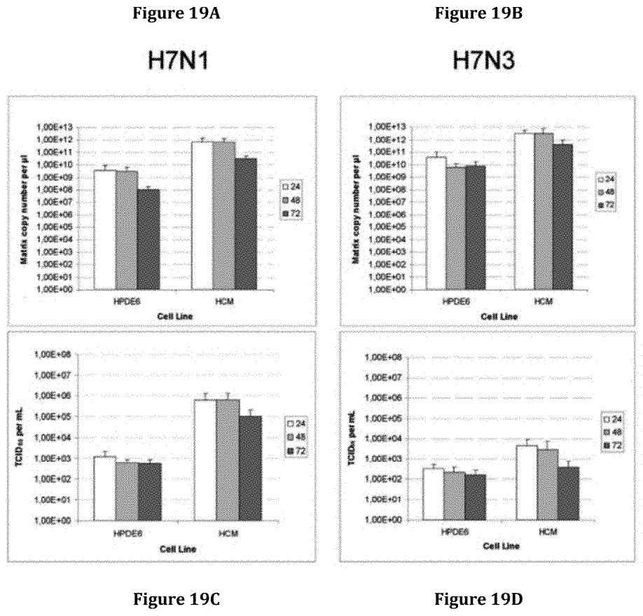

Despite the higher M.O.I used to perform the infections (M.O.I=0.01) avian influenza virus showed lower levels of replication in both pancreatic cell lines compared to the human viruses (FIGS. 19A, 19B, 19C, and 19D), with a trend characterized by steady levels of virus RNA up to 48 hours p.i. and a decrease for both cell lines at 72 hours p.i. Based on the RRT-PCR results, hCM appeared to be more sensitive to avian viruses since the total amount of “M gene” RNA on average resulted 2 logs higher than HPDE6 (FIG. 19A and 19B). This was confirmed also by TCID50 results (FIG. 19C and 19D), in which both viruses reached higher titres in hCM. In the latter, however the H7N1 strain exhibited a higher replication efficacy in compared to H7N3. This result is not reflected in the RRT-PCR results for which comparable amounts of viral RNA were detected for both viruses. No significant differences in the viral replication between the two avian viruses were observed in HPDE6.

### Western Blot Analysis for Detection of Virus Nucleoprotein

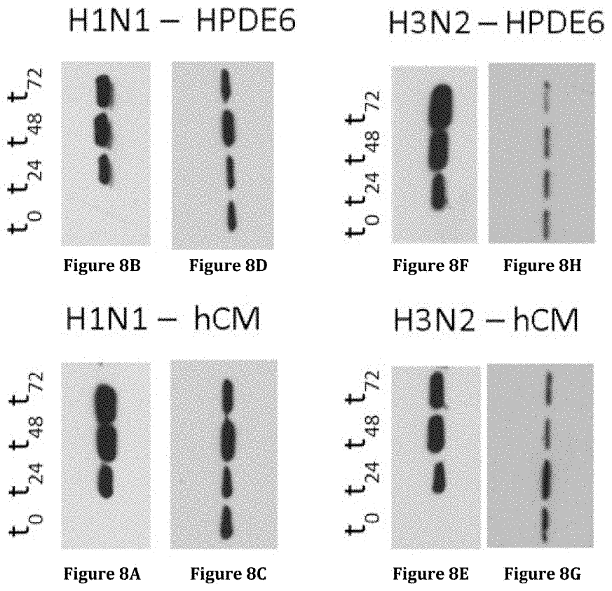

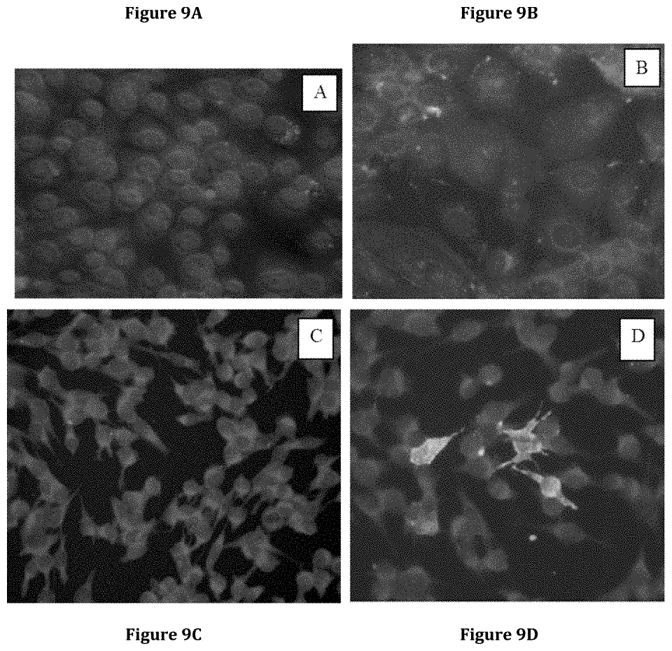

Results of H1N1 and H3N2 influenza virus nucleoprotein in hCM and HPDE6 cell lines are reported in FIGS. 8A, 8B, 8E, and 8F. No differences, depending either on the viral strain or on the cell type, were shown in the trend of NP expression. As expected influenza virus nucleoprotein was not observed at to (before infection), while it was detected at 24 (t₂₄), 48 (t₄₈) and 72 (t₇₂) hours post-infection for both viruses in hCM as well as in HPDE6. Comparing the bands obtained from samples at t₂₄ to those obtained at t₄₈ and t₇₂ an increase in the NP expression was observable. On the other hand the amount of beta actin, used as loading control, was at the same levels in all the samples tested (FIGS. 8C, 8D, 8G, and 8H).

### Immunofluorescence Targeting the NP Protein

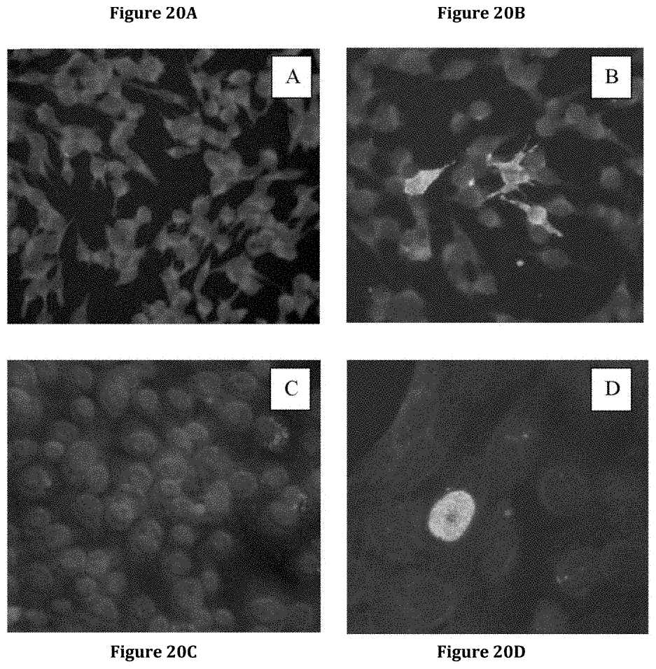

Human influenza virus replication was also detected by a fluorescent signal derived from FITC conjugate in hCM at 24 h post-infection (FIGS. 20A and 20B) for both viruses tested and increased over time at 48 and 72 hours post-infection. No differences were observed between the viral stains tested. The fluorescence signal for both viruses observed at 24 h post-infection in HPDE6 cells (FIGS. 20C and 20D). Also, in this case the number of cells marked continued to increase at 48 and 72 h post-infection, demonstrating the enhancement of the nucleoprotein expression over time (data not shown).

### Susceptibility of Human Pancreatic Islet to Human Influenza A Viruses

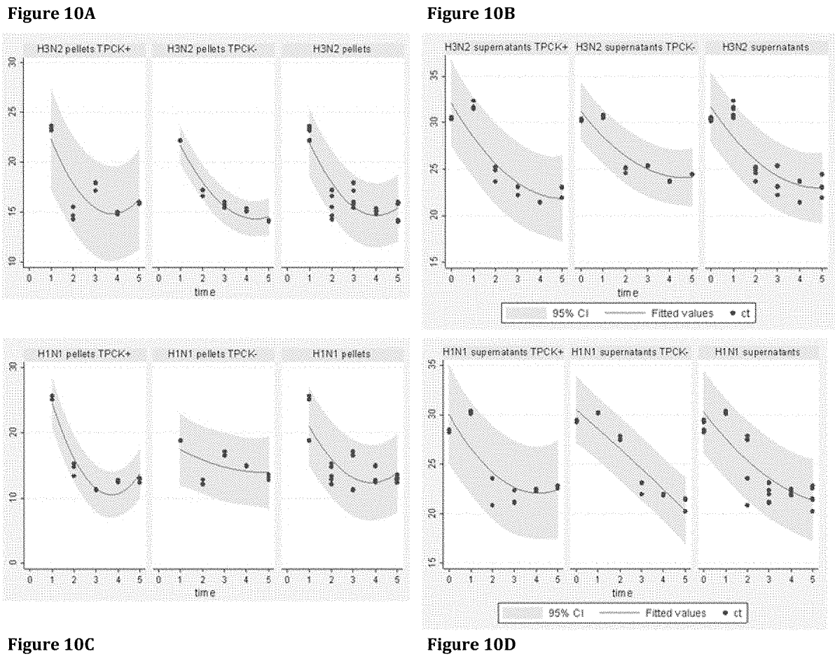

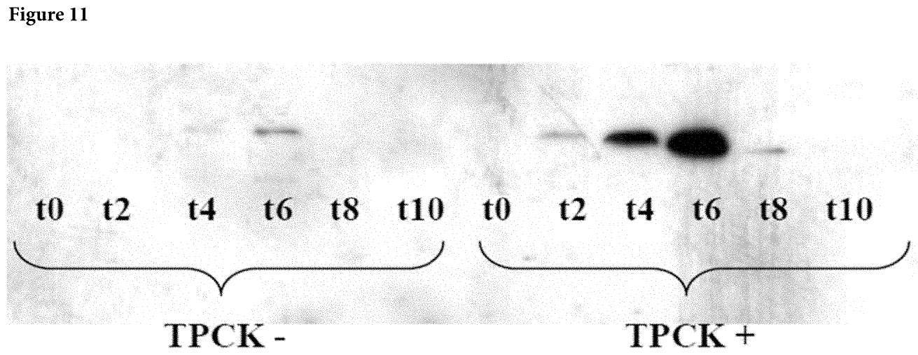

The regression model indicated that the Ct values for both viruses, in presence or in absence of TPCK-trypsin, tested in both in pellets or in supernatant specimens, decreased significantly over time (p<0.05) (FIGS. 10A, 10B, 10C, and 10D). The statistical analysis showed that the virus titer increased over time independently of the virus subtype and type of sample (pellet or supernatant). Interestingly, only for H1N1 pellets and supernatant samples Ct values for the viruses grown with TPCK-trypsin decreased significantly more than those obtained without the exogenous proteases (p<0.01) (FIGS. 10A and 10C). TPCK-trypsin seemed to enhance H3N2 virus growth but the difference did not reach statistical significance (p>0.10) (FIG. 11). The residuals post-estimation analysis indicates that the model used was appropriate (data not shown).

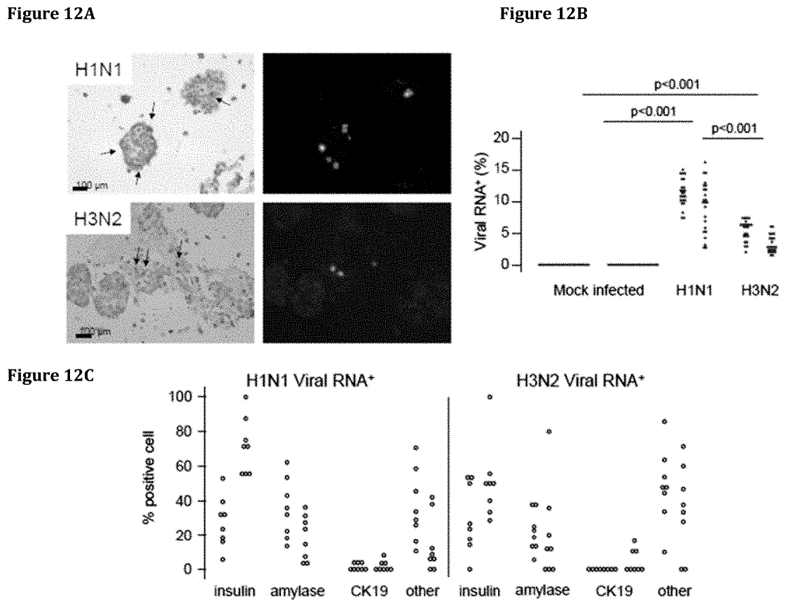

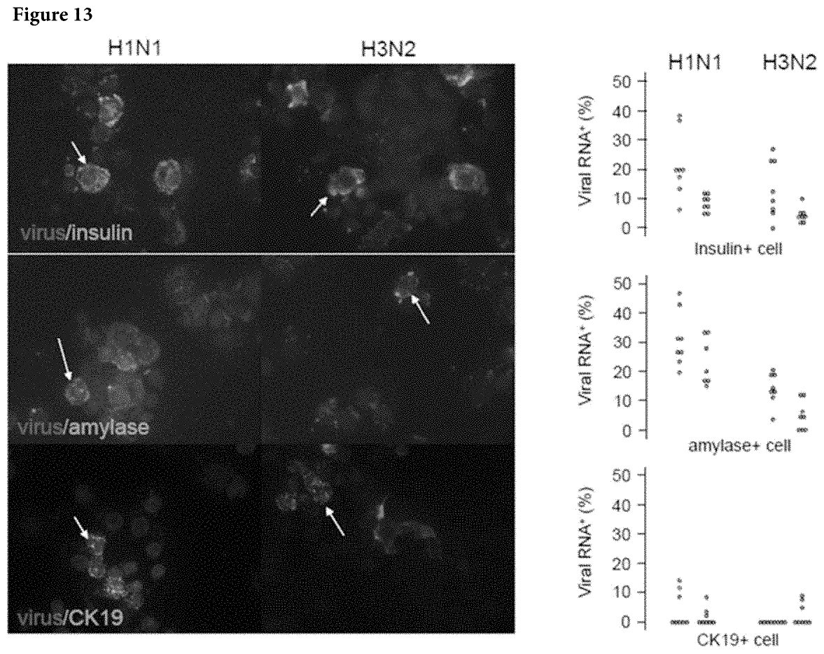

In situ hybridization was performed to visualize viral RNA localized within islet cells. The results clearly demonstrate the presence of viral RNssA both after H1N1 and H3N2 infection (FIG. 12A). Since human islet primary cultures contain both endocrine and exocrine cells a fluorescence-based multiplex in situ hybridization strategy was applied to determine which and how many cells were infected in the islets. For this purpose distinctly labelled probes were combined to analyze viral RNA and insulin, amylase or cytokeratin 19 transcripts simultaneously and, after hybridization, human islets were disaggregated and cells positivity quantified. Five days after infection 0%, 10.8% and 4.3% of total cells resulted positive for viral RNA in mock, H1N1 and H3N2 infected islets, respectively (p<0.001) (FIG. 12B). Of the H1N1 positive cells 49±27% stained positive for insulin, 26±16% for amylase, 1.6±2.4% for CK19 and 25±21% were negative for tested transcripts. Of the H3N2 positive cells 40±23% stained positive for insulin 20±20% for amylase, 2.3+5% for CK19 and 41±45% were negative for tested transcripts (FIG. 12C). On the other hand, of the insulin positive cells 14±10% and 8±8% were positive for viral RNA 5 days after H1N1 and H3N2 infection respectively (p=0.023). Of the amylase positive cell 27±9% and 9±6% were positive for viral RNA after H1N1 and H3N2 infection, respectively (p<0.001). Of the CK19 positive cell 3±4% and 1.3±3% were positive for viral RNA after H1N1 and H3N2 infection, respectively (p=0.36) (FIG. 13).

Modulation of Survival, Insulin Secretion and Innate Immunity in Human Pancreatic Islets Infected with Hwnan Influenza A Viruses In Vitro.

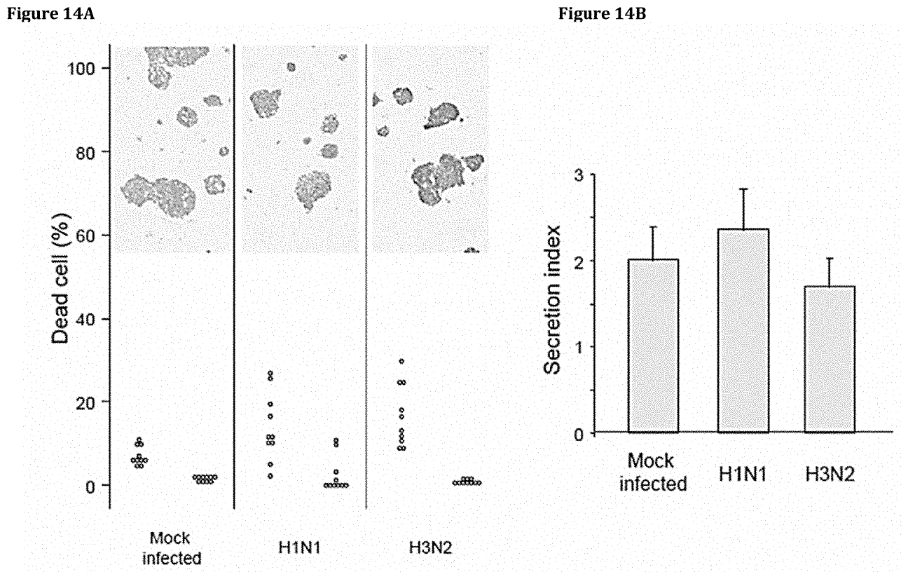

Visual examination of the infected islets by light microscopy and Live/Dead assay revealed no significant cytopathic effect at any time point post-infection (day 0-7). Five days after infection, uninfected cells showed an overall mortality of 3.26%, H3N2 of 5.21% and H1N1 of 7.38% (p=ns vs mock infected cell) (FIGS. 14A and 14B). Moreover exposure of islets to both H1N1 and H3N2 did not affect their ability to respond to high glucose, as tested in a static perfusion system (FIGS. 14A and 14B).

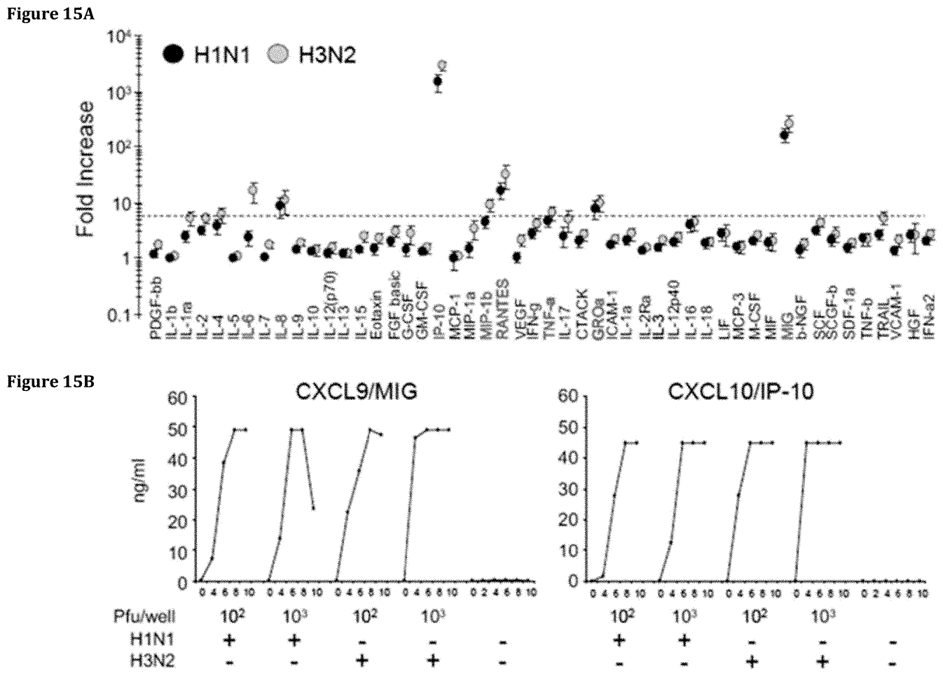

The capability of H1N1 and H3N2 to induce cytokine/chemokines expression m human pancreatic islet was measured using multiplex bead-based assays based on xMAP technology. The parallel wells of human islets (150 islets/well) were infected with HINI and H3N2 at 102 103 pfu/well, or they were mock infected. The culture media supernatant was collected at five time points (0, 4, 6, 8, 10 days) post infection, and assayed for 50 cytokines. With the exception of three (1L-1b, 1L-5, 1L-7) all the cytokines showed detectable expression. In mock infected the highest concentrations were detected for CCL2/MCP1 (max 25,558 pg/ml, day 4), ICAM-1 (max 14,063, day 1 0), CXCL8/IL-8 (max 11,6 pg/ml, day 1 0); IL-6 (8,452 pg/ml, day 4), CXCL1/GRO-α (max 8,581 pg/ml, day 4), VCAM-1 (max 5,566 pg/ml, day 6) VEGF (max 3,225 pg/ml, day 10), SCGF-b (max 1,427 pg/ml, day 6), HGF (max 1,195 pg/ml, day 6). MIF (max 806 pg/ml, day 6), G-CSF (max 794 pg/ml day 6), CXCL9/MIG (max 448 pg/ml, day 6) GM-CSF (max 280 pg/ml, day 4), IL-2Ra (max 230 pg/ml, day 6), IL-12p40 (max 215 pg/ml, day 6), M-CSF (max 212 pg/ml, day 10), LIF (max 185 pg/ml, day 6), CXCL4/SDF1 (max 121 pg/ml, day6) showed lower but consistent expression. CXCL10/IP-10, PDGF-BB, IL-1Ra, IL-12p70, CCL11/Eotaxin, FGFb, CCLS/RANTES, CCL4/MIP-1β, CCL7/MCP-3, IL-3, IL-16, SCF, TRAIL, INFa2, INFg, CCL27/CTAK showed low but consistent expression (max between 10 to 100 pg/ml). Very low (max <10 pg/ml) but detectable expression was present for IL-2, IL-4, IL-9, IL10, IL-13, IL-15, CCL3/MIP-α, TNF-α, IL-17, IL-18, IL1α, β-NGF, TNF-β. Two inflammatory cytokines (IL-6, TNFα) and six inflammatory chemokines (CXCL8/IL-8, CXCL1/GRO-α, CXCL9/MIG, CXCL10/IP-10, CCLS/RANTES, CCL4/MIP-1β) showed over fivefold increase in influenza viruses-infected cell supernatants compared to mock-infected controls (FIG. 15A). Between these the INF-γ inducible chemokines CXCL9/MIG, CXCL10/IP-10 showed the strongest response to H1N1 or H3N2 infection (over one hundred fold increase). Both peaked 6-8 days post infection and showed a stronger response to higher dose of viruses (FIG. 15B).

### Summary of Results

The objective of this work was to assess IAV replication in pancreatic cells and to evaluate its consequence both at cellular level in vitro and at tissue level in vivo. These studies indicate, for the first time, that human influenza A viruses are able to grow in human pancreatic primary cells and cell lines. The addition of exogenous trypsin appears to enhance viral replication, but is surprisingly not essential for viral replication in human pancreatic primary cells and cell lines. The inventors' in vivo results confirmed these findings, where two non-systemic strains of IAVs were able to colonise the pancreas of experimentally infected poults and with metabolic consequences that reflect endocrine and exocrine damage.

The colonisation of the pancreas by IAV has been reported following a number of natural and experimental infections of animals, primarily in birds undergoing both systemic and nonsystemic infection (see references above). However, there is no direct evidence of infection of the pancreas in humans. Here, the inventors have demonstrated for the first time that two non-systemic avian influenza viruses cause severe pancreatitis resulting in a dismetabolic condition comparable with diabetes as it occurs in birds. Literature is available on the clinical implications of endocrine and exocrine dysfunctions of the pancreas in birds, including poultry. Regarding endocrine function, several studies indicate that with a total pancreatectomy birds suffer severe hypoglycaemic crisis leading to death [103]. If a residual portion of the pancreas as small as 1% of the pancreatic mass is left in situ, a transient (or reversible) hyperglycaemic condition is observed in granivorous birds, in which, normal glycemia is re-established within a couple of weeks [104,105]. This indicates that the pancreatic tissue of birds has significant compensatory potential and is also influenced by the fact that there is evidence towards the presence of some endocrine tissue able to secrete insulin outside the pancreas [106]. Insulin is the dominant hormone in the well-fed bird, while glucagon is the dominant hormone in the fasting bird. In this experiment, which was carried out with food ad libitum, damage of the endocrine component of the pancreas, would likely manifest itself with hyperglycemia.

Regarding exocrine function, pancreatitis in birds is characterised by malaise, reluctance to feed, enteritis and depression. Intra-vitam investigations are based on increased haematic lipase concentration [105]. In this study pancreatitis was evaluated by measuring the lipase concentration in the blood stream, and by histopathologic examination of pancreas collected at different time points. As it occurs in mammals, pancreatic damage determined a rapid increase of the haematic lipase levels which was transient and the values returned to normal by day 15 p.i. Interestingly, the birds which had shown the increased lipase levels in the blood and thus supposedly the most severe pancreatic damage, exhibited in the subsequent days high blood glucose levels, which only in a few cases persisted until the termination of the experiment. This is in-keeping with the clinical and metabolic presentation of diabetes in birds. The histological investigations clearly indicate viral replication in the exocrine portion of the pancreas, resulting in fibrosis and disruption of the organ's architecture. While it is clear that both isolates under study replicated extensively in the acinar component of the pancreas, the inventors were unable to determine whether viral replication also occurred in the islets. Based on these results, the inventors suggest that influenza virus infection caused severe acute pancreatitis which has impaired both the endocrine and exocrine functions.

Current knowledge on influenza replication indicate that influenza viruses which do not exhibit a multibasic cleavage site of the HA protein do not become systemic. However, in the in vivo experiments the virus reached the pancreas, and the inventors have surprisingly detected viral RNA on day 3 post infection from the blood in 2/20 (Group A- H7N1) and 4/20 (Group B-H7N3) infected turkeys. The inventors postulate that, following replication in target organs such as the lung and the gut, in some individuals, a small amount of virus reaches the bloodstream and thus the pancreas. Although the detected Ct values detected indicate low levels of viral RNA, this often resulted in the development of pancreatitis (detected in vivo by hyperlipasemia). This in turn, in the experimental model has resulted in an hyperglycaemic condition, consistent with the presentation of diabetes in granivorous birds.

The results of the in vitro experiments show that all IAVs tested, both of avian (H1N1 and H7N3) and of human origin (HINI Caledonia/20/99 and H3N2 A/Wisconsin/67/2005) are able to grow in established pancreatic cell lines and in pancreatic islets. Viral replication occurs both in cells of endocrine and exocrine origin. These investigations also show that both alpha-2-3 and alpha-2-6 receptors are present in pancreatic cells, indicating that both human and avian influenza viruses could find suitable receptors in this organ. The human viruses used in this study did not induce a significant mortality of islet cells, and insulin secretion did not appear to be affected by infection in this system. On the other hand, it was clear from the cytokine expression profile that IAV infection is able to induce a strong pro-inflammatory program in human pancreatic islets. The INF-gamma-inducible chemokines MIG/CXCL9/and IP-10/CXCL10 showed the highest increase after infection. Also huge amounts of RANTES/CCL5, MIP1b/CCL4, Groa/CXCL1, IL8/CXCL8, TNFa and IL-6 were released. Of interest, many of these factors were described as key mediators in the pathogenesis of type 1 diabetes [107].

Recently 1P10/CXCL10 was identified as the dominant chemokine expressed in vivo in the islet environment of prediabetic animals and type 1 diabetic patients whereas RANTES/CCL5 and MIG/CXCL9 proteins were present at lower levels in the islets of both species [108]. The chemokine IP-10/CXCL10 attracts monocytes, T lymphocytes and NK cells, and islet-specific expression of CXCL10 in a mouse model of autoimmune diabetes caused by viruses [rat insulin promotor (RIP)-LMCV] accelerates autoimmunity by enhancing the migration of antigenspecific lymphocytes [109]. This is in keeping with bother findings in which neutralization of IP-10/CXCL10 [110] or its receptor (CXCR3) [111] prevents autoimmune disease in the same mouse model (RIP-LCMV). Studies in NOD mice have demonstrated elevated expression of IP-10/CXCL10, mRNA and/or protein in pancreatic islets during the prediabetic stage [112]. Increased levels of MIP1b/CCL4 and IP-10/CXCL10 are present in the serum of patients who have recently been diagnosed as having type 1 diabetes [113,114].

The inventors propose that, if influenza virus finds its way to the pancreas, either through viraemia, as detected in human patients [115,116, 117], or through reflux from the gut through the pancreatic duct, the virus would find a permissive environment. Here, the virus would encounter appropriate cell receptors and susceptible cells belonging to both the endocrine and exocrine component of the organ. Viral replication would result in cell damage due to the activation of a cytokine storm similar to the one associated with various conditions linked to diabetes. Thus the inventors believe that influenza infections may lead to pancreatic damage resulting in acute pancreatitis and/or onset of type 1 diabetes.

### Conclusion

These results provide the first evidence of a causal link between influenza virus infection and the development of type 1 diabetes and/or pancreatitis. This causal link between infection and type 1 diabetes and/or pancreatitis provides various therapeutic, prophylactic and diagnostic opportunities.

The above description of preferred embodiments of the invention has been presented by way of illustration and example for purposes of clarity and understanding. It is not intended to be exhaustive or to limit the invention to the precise forms disclosed. It will be readily apparent to those of ordinary skill in the art in light of the teachings of this invention that many changes and modifications may be made thereto without departing from the spirit of the invention. It is intended that the scope of the invention be defined by the appended claims and their equivalents.

### REFERENCES (THE CONTENTS OF WHICH ARE HEREBY INCORPORATED IN FULL BY REFERENCE)

[1] Hyoty & Talyor (2002) Diabetologia 45:13-61.

[2] Jun & Yoon (2001) Diabetologia 44(3):271-85.

[3] Blum, A. et al. 2010. Isr. Med. Assoc. J. 12:640-641.

[4] Calore, E. E. et al. 2011. Pathol. Res. Pract. 207:86-90.

[5] Rimmelzwaan, G. F. et al. 2006. Am. J Pathol. 168:176-83.

[6] Herlocher et al. (2004) J Infect Dis 190(9):1627-30.

[7] Le et al. (2005) Nature 437(7062):1108.

[8] Nikolaeva-Glomb & Galabov (2004) Antiviral Res 62(1):9-19

[9] Abe et al. (2001) EurJ Pharm Sci Apr; 13(1):61-9

[10] Vaccines. (eds. Plotkin & Orenstein). 4th edition, 2004, ISBN: 0-7216-9688-0.

[11] WO96/37624.

[12] WO98/46262.

[13] WO2007/085969.

[14] WO02/28422.

[15] WO02/067983.

[16] WO02/074336.

[17] WO01/21151.

[18] WO02/097072.

[19] WO2005/113756.

[20] Huckriede et al. (2003) Methods Enzymol 373:74-91.

[21] Treanor et al. (1996) J Infect Dis 173:1467-70.

[22] Keitel et al. (1996) Clin Diagn Lab Immunol 3:507-10.

[23] Rota et al. (1992) J Gen Virol 73:2737-42.

[24] GenBank sequence GI:325176.

[25] Hoffmann et al. (2002) Vaccine 20:3165-3170.

[26] Subbarao et al. (2003) Virology 305:192-200.

[27] Liu et al. (2003) Virology 314:580-590.

[28] Ozaki et al. (2004) J Viral. 78:1851-1857.

[29] Webby et al. (2004) Lancet 363:1099-1103.

[30] WO00/60050.

[31] WO01/04333.

[32] U.S. Pat. No. 6,649,372.

[33] Neumann et al. (2005) Proc Natl Acad Sci USA 102:16825-9.

[34] WO2006/067211.

[35] WO01/83794.

[36] Hoffmann et al. (2000) Virology 267(2):310-7.

[37] Herlocher et al. (2004) J Infect Dis 190(9):1627-30.

[38] Le et al. (2005) Nature 437(7062): 1108.

[39] Kistner et al. (1998) Vaccine 16:960-8.

[40] Kistner et al. (1999) Dev Biol Stand 98:101-110.

[41] Bruhl et al. (2000) Vaccine 19:1149-58.

[42] Pau et al. (200 1) Vaccine 19:2716-21.

[43] http://www.atcc.org/

[44] http://locus.umdnj.edu/ [45] WO03/076601.

[46] WO2005/042728.

[47] WO03/043415.

[48] WO01/85938.

[ 49] WO2006/108846.

[50] WO97/37000.

[51] Brands et al. (1999) Dev Biol Stand 98:93-100.

[52] Halperin et al. (2002) Vaccine 20:1240-7.

[53] Tree et al. (2001) Vaccine 19:3444-50.

[54] EP-A-1260581 (WO01/64846).

[55] WO2006/071563.

[56] WO2005/113758.

[57] WO97/37001.

[58] WO2006/027698.

[59] EP-B-0870508.

[60] U.S. Pat. No. 5,948,410.

[61] Lundblad (2001) Biotechnology and Applied Biochemistry 34:195-197.

[62] Guidance for Industry: Bioanalytical Method Validation. U.S. Department of Health and Human Services Food and Drug Administration Center for Drug Evaluation and Research (CDER) Center for Veterinary Medicine (CVM). May 2001.

[63] Ji et al. (2002) Biotechniques. 32:1162-7.

[64] Briggs (1991) J Parenter Sci Technol. 45:7-12.

[65] Lahijani et al. (1998) Hum Gene Ther. 9:1173-80.

[66] Lokteff et al. (2001) Biologicals. 29:123-32.

[67] Rodacki et al. (2007) Diabetes. 56(1): 177-85

[68] Qin et al. (2011) Diabetes. 60(3): 857-866

[69] Harrison and Honeyman (1999) Diabetes. 48(8): 1501-1507

[70] Geysen et al. (1984) PNAS USA 81:3998-4002.

[71] Carter (1994) Methods Mol Biol 36:207-23.

[72] Jameson, B A et al. 1988, CABIOS 4(1): 181-186.

[73] Raddrizzani & Hammer (2000) Brief Bioinform 1 (2):179-89.

[74] De Lalla et al. (1999) J Immunol. 163:1725-29.

[75] Brusic et al. (1998) Bioinformatics 14(2):121-30

[76] Meister et al. (1995) Vaccine 13(6):581-91.

[77] Roberts et al. (1996) AIDS Res Hum Retroviruses 12(7):593-61 0.

[78] Maksyutov & Zagrebelnaya (1993) Comput Appl Biosci 9(3):291-7.

[79] Feller & de la Cruz (1991) Nature 349(6311):720-1.

[80] Hopp (1993) Peptide Research 6:183-190.

[81] Welling et al. (1985) FEES Lett. 188:215-218.

[82] Davenport et al. (1995) Immunogenetics 42:392-297.

[83] Farrell (1998) RNA Methodologies (Academic Press; ISBN 0-12-249695-7).

[84] EP-B-0509612.

[85] EP-B-0505012.

[86] Fille et al. (1997) Biotechniques 23:34-36.

[87] Gennaro (2000) Remington: The Science and Practice of Pharmacy. 20th edition, ISBN: 0683306472.

[88] Harder, T. C. and T. W. Vahlenkamp. 2010. Vet. Immunol. Immunopathol. 134:54-60

[89] Kuiken, T. et al. 2004. Science. 306:241.

[90] Capua, I. et al. 2009. Springer.

[91] Tanimura, N. et al. 2006. Vet. Pathol. 43:500-509.

[92] Teifke, J. P. et al. 2007. Vet. Pathol. 44:137-143.

[93] Abolnik, C. B. Z. et al. 2009. Influenza Other Respi Viruses. 3:63-68.

[94] Bertran, K. et al. 2011. Vet. Res. 4 2:24.

[95] Shinya, K. et al. 1995. Avian Pathol. 24:623-632.

[96] Mutinelli, F. et al. 2003. Avian Dis. 47:844-848.

[97] Okamatsu, M. et al. 2007. Vet. Microbial. 124:-46.

[98] Spackman, E. et al. 2002. J Clin. Microbial. 40:3256-3260.

[99] Baroni, M. G. et al. 1999. J Endocrinol. 161:59-68.

[100] Ouyang, H. et al. 2000. Am. J Pathol. 157:1623-1631.

[101] Matrosovich, M., T. et al. 2006. Viral. J. 2 Aug. 31;3:63 3:63.

[102] Wakeley, P. R. et al. 2006. Dev. Biol. (Basel). 126:227-36; discussion 326-7.

[103] Hazelwood R. L. 2000. Pancreas., p. 539-555. In Academic Press (ed.), Sturkie's avian physiology., 5th edition. ed., . Whittow G C, editor, San Diego.

[104] Laurent, F., and P. Mialhe. 1978. Diabetologia. 15:313-321.

[105] Walter E. Hoffmann & Philip F. Solter. 2008. Diagnostic enzymology of domestic animals., p. 365-366. In Elsevier Inc. All rights reserved (ed.), Clinical Biochemistry of Domestic Animals (Sixth Edition) vol. 12. J. Jerry Kaneko, John W. Harvey and Michael L. Bruss.

[106] Colca, J. R., and R. L. Hazelwood. 1982. J. Endocrinol. 92:317-326.

[107] Eizirik, D. L. et al. 2009. Nat. Rev. Endocrinol. 5:219-226.

[108] Sarkar, S. A. et al. 2012. Diabetes. 61:436-446.

[109] Rhode, et al. 2005. J. Immunol. 175:16-24.

[110] Christen, U. et al. 2003. J. Immunol. 171:6838-6845.

[111] Frigerio, S. eta;. 2002. Nat. Med. 8:1414- 1420.

[112] Cardozo, A. K. et al. 2003. Diabetologia. 46:255-266.

[113] Hanifi-Moghaddam, P. et al. 2006. Diabet. Med. 23:156-163.

[114] Shimada, A. et al. 2001. Diabetes Care. 24:510-515.

[115] Likos, A. M. et al. 2007. Transfusion. 47:1080-1088.

[116] Oughton, M. et al. 2011. Diagn. Microbiol. Infect. Dis. 70:213-217.

[117] Tse, H. et al. 2011. PLoS One. 6:e22534.

1-20. (canceled)

21. A method of treating type 1 diabetes in a patient comprising:
selecting the patient in need of treatment for type 1 diabetes, and
vaccinating the patient with an immunogenic composition, wherein the vaccination of the patient prevents or reduces the severity of influenza infection, thereby reducing the effects of the influenza infection on the type 1 diabetes.

22. The method of claim 21, further comprising treating the patient with at least one of the following treatments selected from the group consisting of islet transplantation, transplantation of beta cell precursors, and stem cells.

23. The method of claim 21, wherein the immunogenic composition comprises an adjuvant.

24. The method of claim 23, wherein the adjuvant is MF59.

25. The method of claim 21, wherein the subject is a child.

26. The method of claim 21, wherein levels of CXCL9/MIG are lowered following vaccination.

27. The method of claim 21, wherein levels of CXCL10/IP-10 are lowered following vaccination.

28. The method of claim 21, wherein levels of CCL5/RANTES, CCL4/MIP1b, CXCL1/Groa, CXCL8/IL8, TNFa, and IL-6 are lowered following vaccination.

29. A method of treating pancreatitis in a patient comprising:
selecting the patient in need of treatment for pancreatitis, and vaccinating the patient with an immunogenic composition, wherein the vaccination of the patient prevents or reduces the severity of influenza infection, thereby reducing the effects of the influenza infection on the pancreatitis.

30. The method of claim 29, further comprising treating the patient with at least one of the following treatments selected from the group consisting of islet transplantation, transplantation of beta cell precursors, and stem cells.

31. The method of claim 29, wherein the immunogenic composition comprises an adjuvant.

32. The method of claim 31, wherein the adjuvant is MF59.

33. The method of claim 29, wherein the subject is a child.

34. The method of claim 29, wherein levels of CXCL9/MIG are lowered following vaccination.

35. The method of claim 29, wherein levels of CXCL10/IP-10 are lowered following vaccination.

36. The method of claim 29, wherein levels of CCL5/RANTES, CCL4/MIP1b, CXCL1/Groa, CXCL8/IL8, TNFa, and IL-6 are lowered following vaccination.
